(19) United States
(12) Patent Application Publication
(10) Pub. No.: US 2025/0074933 A1
(43) Pub. Date: Mar. 6, 2025

(54) DOUBLE-STRANDED RNA HAVING NUCLEOTIDE ANALOG

(71) Applicant: Rona Bioscience, Limited, Hong Kong (CN)

(72) Inventors: Jinyu Huang, Shanghai (CN); Hongli Guo, Shanghai (CN)

(73) Assignee: Rona Bioscience, Limited, Hong Kong (CN)

(21) Appl. No.: 18/726,560

(22) PCT Filed: Jan. 4, 2023
(86) PCT No.: PCT/CN2023/070382

§ 371 Date: Jul. 3, 2024

(30) Foreign Application Priority Data
Jan. 5, 2022 (CN) 202210005852.4
Aug. 8, 2022 (CN) 202210948347.3
Dec. 14, 2022 (CN) 202211609905.X

(51) Int. Cl. C07H 19/167; A61K 48/00; C12N 15/113

(52) U.S. Cl. CPC C07H 19/167; A61K 48/005; C12N 15/113; C12N 2310/11

(57) ABSTRACT

Provided in the present invention is a double stranded RNA having a nucleotide analog. The double-stranded RNA of the present invention shows one or more of enhanced stability reduced off-target toxicity and enhanced efficiency.

### FIELD OF TECHNOLOGY

The present invention belongs to the field of medicine and specifically relates to a double-stranded RNA having a nucleotide analog.

### BACKGROUND

RNA interference is a phenomenon of efficient and specific degradation of a target mRNA induced by a double-stranded RNA (dsRNA). Incorporation of thermally unstable nucleotides, such as glycerol nucleic acid (ONA), into a seed region of an antisense strand of the double-stranded RNA is conducive to improving interference efficiency and reducing off-target toxicity, with reference to, for example, PCT publication No. WO2018098328A1. However, as described in Schlegel et al. Nucleic Acids Research (2021), compared with GNA-A or GNA-T, the incorporation of GNA-G or GNA-C in the center of the double-stranded RNA will lead to reduction of the stability of the double-stranded RNA.

Therefore, a nucleotide analog having enhanced stability when the double-stranded RNA is incorporated is required to be developed in the field.

### SUMMARY

The present invention solves the above problems by providing a novel nucleotide analog

In one aspect, the present invention relates to a nucleotide dimer as shown in Formula (A),

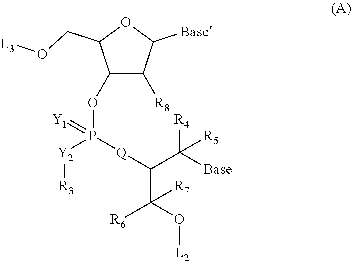

where,
L₂ is H or P₂;
L₃ is H or P₃;
Q is —X— or —X—O—;
X is a chemical bond, —(CR₁R₂)ₘ—, or —CR₁═CR₂;
Y₁ is O, S, or NR;
Y₂ is O, S, or a chemical bond;
R₁, R₂, R₄, R₅, R₆ and R₇ are independently selected from H, D, halogen, CN, C₁₋₆ alkyl, C₁₋₆ haloalkyl, C₂₋₆ alkenyl, or C₂₋₆ alkynyl, and are optionally substituted with 1, 2, 3, 4, or 5 R′(s),
R₃ is selected from H, C₁₋₆ cyanoalkyl, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl, and is optionally substituted with 1, 2, 3, 4, or 5 R′(s);
R₈ is selected from H, D, OH, halogen, C₁₋₆ alkyl, C₁₋₆ haloalkyl, or C₁₋₆ alkoxyl;
Base and Base′ are independently selected from H, a modified or unmodified base, or a leaving group.
P₂ is selected from a reactive phosphorus group, preferably —P(OCH₂CH₂CN)(N(iPr)₂),
P₃ is selected from a hydroxyl protective group, preferably DMTr;
R is selected from H, C₁₋₆ alkyl, or C₁₋₆ haloalkyl;
R′ is selected from D, halogen, OH, CN, NH₂, C₁₋₆ alkyl, C₁₋₆ haloalkyl, C₂₋₆ alkenyl, C₂₋₆ alkynyl, C₃₋₁₀ cycloalkyl, 3- to 10-membered heterocyclyl, C₆₋₁₀ aryl, or 5- to 14-membered heteroaryl, and
m is selected from 1, 2, 3, 4, or 5.

In another aspect, the present invention relates to a double-stranded RNA molecule or a pharmaceutically acceptable salt, tautomer or stereoisomer thereof. The double-stranded RNA molecule includes a sense strand and an antisense strand, where each strand has 14 to 30 nucleotides, the antisense strand has a sequence that is fully complementary to the sense strand and a target mRNA, and has an ability to induce degradation of the target mRNA, and the antisense strand includes one or more of a nucleotide monomer as shown in Formula (IV):

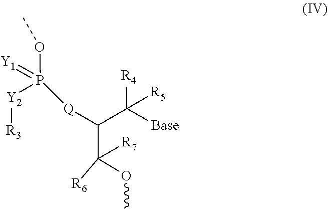

where,
the nucleotide monomer is connected from [symbol] to [symbol] in the order of 5′=>3′; and
various groups are as defined in the context.

In another aspect, the present invention relates to a nucleic acid molecule. A nucleotide sequence of the nucleic acid molecule includes one or more of the nucleotide monomer as described herein and/or the nucleotide dimer as described herein.

In another aspect, the present invention relates to a pharmaceutical composition. The pharmaceutical composition includes the double-stranded RNA molecule as described herein and a pharmaceutically acceptable carrier or excipient.

In another aspect, the present invention relates to a kit. The kit includes the double-stranded RNA molecule as described herein.

In another aspect, the present invention relates to a method for inhibiting expression of a target gene in a cell. The method includes a step of introducing the double-stranded RNA molecule as described herein into the cell.

In another aspect, the present invention relates to a method for inhibiting expression of a target gene in a cell. The method includes expressing the double-stranded RNA molecule as described herein in the cell.

After a nucleotide of the present invention is incorporated into an antisense strand of a dsRNA, a resulting double-stranded RNA exhibits one or more of enhanced stability, reduced off-target toxicity, and enhanced effectiveness.

### Definition

### Chemical Definition

Definitions of specific functional groups and chemical terms are described in more detail below.

When a numerical range is listed, it is agreed to include each value and subranges within the range. For example, the “C₁₋₆ alkyl” includes C₁ alkyl, C₂ alkyl, C₃ alkyl, C₄ alkyl, C₅ alkyl, C₆ alkyl, C₁₋₆ alkyl, C₁₋₅ alkyl, C₁₋₄ alkyl, C₁₋₃ alkyl, C₁₋₂ alkyl, C₂₋₆ alkyl, C₂₋₅ alkyl, C₂₋₄ alkyl, C₂₋₃ alkyl, C₃₋₆ alkyl, C₃₋₅ alkyl, C₃₋₄ alkyl, C₄₋₆ alkyl, C₄₋₅ alkyl, and C₅₋₆ alkyl

The “C₁₋₆ alkyl” refers to a straight-chain or branched-chain saturated hydrocarbon group having 1 to 6 carbon atoms. In some embodiments, C₁₋₄ alkyl and C₁₋₂ alkyl are preferred Examples of the C₁₋₆ alkyl include: methyl (C₁), ethyl (C₂), n-propyl (C₃), isopropyl (C₃), n-butyl (C₄), tert-butyl (C₄), sec-butyl (C₄), isobutyl (C₄), n-pentyl (C₅), 3-pentyl (C₅), pentyl (C₅), neopentyl (C₅), 3-methyl-2-butyl (C₅), tert-pentyl (C₅), and n-hexyl (C₆). The term “C₁₋₆ alkyl” further includes heteroalkyl, in which one or more (such as 1, 2, 3, or 4) carbon atoms are substituted with a heteroatom (such as oxygen, sulfur, nitrogen, boron, silicon, or phosphorus). An alkyl group can be substituted with one or more substituents, for example, substituted with 1 to 5 substituents, 1 to 3 substituents, or 1 substituent. Common alkyl abbreviations include: Me(—CH₃), Et(—CH₂CH₃), iPr(—CH(CH₃)₂), nPr(—CH₂CH₂CH₃), n-Bu(—CH₂CH₂CH₂CH₃), or i-Bu(—CH₂CH(CH₃)₂).

The “C₂₋₆ alkenyl” refers to a straight-chain or branched-chain hydrocarbon group having 2 to 6 carbon atoms and at least one carbon-carbon double bond. In some embodiments, C₂₋₄ alkenyl is preferred. Examples of the C₂₋₆ alkenyl include: vinyl (C₄), 1-propenyl (C₅), 2-propenyl (C₅), 1-butenyl (C₄), 2-butenyl (C₄), butadienyl (C₄), pentenyl) (C₅), pentadienyl (C₅), hexenyl (C₆), etc. The term “C₂₋₆ alkenyl” father includes heteroalkenyl, in which one or more (such as 1, 2, 3, or 4) carbon atoms are substituted with a heteroatom (such as oxygen, sulfur, nitrogen, boron, silicon, or phosphorus). An alkenyl group can be substituted with one or more substituents, for example, substituted with 1 to 5 substituents, 1 to 3 substituents, or 1 substituent.

The “C₂₋₆ alkynyl” refers to a straight-chain or branched-chain hydrocarbon group having 2 to 6 carbon atoms, at least one carbon-carbon triple bond, and optionally one or more carbon-carbon double bonds. In some embodiments, C₂₋₄ alkynyl is preferred Examples of the C₂₋₆ alkynyl include, but are not limited to: acetonyl (C₂), 1-propynyl (C₃), 2-propynyl (C₃), 1-butynyl (C₄), 2-butynyl (C₄), pentynyl (C₅), hexynyl (C₆), etc. The term “C₂₋₆ alkynyl” further includes heteroalkynyl, in which one or more (such as 1, 2, 3, or 4) carbon atoms are substituted with a heteroatom (such as oxygen, sulfur, nitrogen, boron, silicon, or phosphorus). An alkynyl group can be substituted with one or more substituents, for example, substituted with 1 to 5 substituents, 1 to 3 substituents, or 1 substituent.

“C₁₋₆ alkylene” refers to a divalent group formed by removing another hydrogen of C₁₋₆ alkyl, and can be substituted or unsubstituted. In some embodiments, C₁₋₄ alkylene, C₂₋₄ alkylene, and C₁₋₃ alkylene are preferred. Unsubstituted alkylene includes, but is not limited to: methylene (—CH₂—), ethylidene (—CH₂CH₂—), propylidene (—CH₂CH₂CH₂—), butylidene (—CH₂CH₂CH₂CH₂—), pentylidene (—CH₂CH₂CH₂CH₂CH₂—), hexylidene (—CH₂CH₂CH₂CH₂CH₂CH₂—), etc. Exemplary substituted alkylene, such as, alkylene substituted with one or more alkyls (methyl), includes, but is not limited to: substituted methylene (—CH(CH₃)—, —C(CH₃)₂—), substituted ethylidene (—CH(CH₃)CH₂—, —CH₂CH(CH₃)—, —C(CH₃)₂CH₂—, —CH₂C(CH₃)₂), substituted propylidene (—CH(CH₃)CH₂CH₂—, —CH₂CH(CH₃)CH₂—, —CH₂CH₂CH(CH₃)—, —(CH₃)₂CH₂CH₂—, —CH₂C(CH₃)₂CH₂—, —CH₂CH₂C(CH₃)₂—), etc.

The “halo” or “halogen” refers to fluorine (F), chlorine (CD), bromine (Br), and iodine (I).

Therefore, the “C₁₋₆ haloalkyl” refers to the “C₁₋₆ alkyl” that is substituted with one or more halogen groups. In some embodiments. C₁₋₄ haloalkyl is particularly preferred, and C₁₋₂ haloalkyl is more preferred. Exemplary haloalkyl includes, but is not limited to: —CF₃, —CH₂F, —CHF₂, —CH₂CH₂F, —CH₂CHF₂, —CF₂CF₃, —CCl₃, —CH₂Cl, —CHCl₂, 2,2,2-trifluoro-1,1-dimethyl-ethyl, etc. A haloalkyl group can be substituted at any available connection point with, for example, 1 to 5 substituents, 1 to 3 substituents, or 1 substituent.

The “C₁₋₆ alkoxyl” refers to an —O—R group, where R is defined as the “C₁₋₆ alkyl” and the “C₁₋₆ haloalkyl” above.

The “C₁₋₆ cyanoalkyl” refers to —C₁₋₆ alkylene-CN, where the “C₁₋₆ alkylene” is as defined above. In some embodiments, C₁₋₄ cyanoalkyl is particularly preferred, and C₁₋₂ cyanoalkyl is more preferred, such as cyanoethyl (—CH₂CH₂CN).

The “C₃₋₁₀ cycloalkyl” refers to a non-aromatic cyclic hydrocarbon group having 3 to 10 cyclic carbon atoms and zero heteroatom. In some embodiments, C₄₋₇ cycloalkyl and C₃₋₆ cycloalkyl are particularly preferred, and C₅₋₆ cycloalkyl is more preferred. The cycloalkyl further includes a ting system in which a cycloalkyl ring is fused with one or more aryls or heteroaryls, where a connection point is on the cycloalkyl ring, and in such case, the number of carbon continuously represents the number of carbon in the cycloalkyl system. Exemplary cycloalkyl includes, but is not limited to: cyclopropyl (C₃), cyclopropenyl (C₃), cyclobutyl (C₃), cyclobutenyl (C₄), cyclopentyl (C₄), cyclopentenyl (C₅), cyclohexyl (C₅), cyclohexenyl (C₆); cyclohexadienyl (C₆), cycloheptyl (C₇), cycloheptenyl (C₇), cycloheptadienyl (C₇), cycloheptatrienyl (C₇), etc. A cycloalkyl group can be substituted with one or more substituents, for example, substituted with) to 5 substituents, 1 to 3 substituents, or 1 substituent.

The “3- to 10-membered heterocyclyl” refers to a group of a 3- to 10-membered non-aromatic ring system having cyclic carbon atoms and 1 to 5 cyclic heteroatoms, where each heteroatom is independently selected from nitrogen, oxygen, sulfur, boron, phosphorus, and silicon. In heterocyclyl containing one or more nitrogen atoms, a connection point may be a carbon or nitrogen atom as long as the valence is acceptable. In some embodiments, 4- to 10-membered heterocyclyl is preferred, which is a 4- to 10-membered non-aromatic ring system having cyclic carbon atoms and 1 to 5 cyclic heteroatoms, in some embodiments, 3- to 8-membered heterocyclyl is preferred, which is a 3- to 8-membered non-aromatic ring system having cycle carbon atoms and 1 to 4 cyclic heteroatoms; 3- to 6-membered heterocycyl) is preferred, which is a 3- to 6-membered non-aromatic ring system having cycle carbon atoms and 1 to 3 cyclic heteroatoms; 4- to 7 membered heterocyclyl is preferred, which is a 4- to 7-membered non-aromatic ring system having cyclo carbon atoms and 1 to 3 cyclic heteroatoms; and 5- to 6-membered heterocyclyl is more preferred, which is a 5- to 6-membered non-aromatic ring system having cyclic carbon atoms and 1 to 3 cycle heteroatoms. The heterocyclyl further includes a ring system in which a heterocyclyl ring is fused with one or more cycloalkyls, where a connection point is on a cycloalkyl ring, or a ring system in which a heterocyclyl ring is fused with one or more aryls or heteroaryls, where a connection point is on the heterocyclyl ring, and in such case, the number of ring members continuously represents the number of ring members in the heterocyclyl ring system. Exemplary 3-membered heterocyclyl containing a heteroatom includes, but is not limited to: azacyclopropanyl, oxacyclopropanyl, and thiorenyl. Exemplary 4-membered heterocycyl) containing a heteroatom includes, but is not limited to: azacyclobutanyl, oxacyclobutanyl, and thiacyclobutanyl. Exemplary 5-membered heterocyclyl containing a heteroatom includes, but is not limited to: tetrahydrofuranyl, dihydrofuranyl, tetrahydrothiophenyl, dihydrothiophenyl, pyrrolidinyl, dihydropyrrolyl, and pyrrol-2,5-dione. Exemplary 5-membered heterocyclyl containing two heteroatoms includes, but is not limited to: dioxolanyl, oxasulfuranyl, disulfuranyl, and oxazolidin-2-one. Exemplary 5-membered heterocyclyl containing three heteroatoms includes, but is not limited to triazolinyl, oxadiazolinyl, and thiadiazolinyl. Exemplary 6-membered heterocyclyl containing one heteroatom includes, but is not limited to: piperidyl, tetrahydropyranyl, dihydropyridyl, and thienyl. Exemplary 6-membered heterocyclyl containing two heteroatoms includes, but is not limited to: piperazinyl, morpholinyl, dithianyl, and dioxanyl. Exemplary 6-membered heterocyclyl containing three heteroatoms includes, but is not limited to: triazinanyl. Exemplary 7-membered heterocyclyl containing one heteroatom includes, but is not limited to azepanyl, oxepanyl, and thiepanyl. Exemplary 5-membered heterocyclyl fused with a C₆ aryl ring (herein, also referred to as 5,6-bicylic heterocyclyl) includes, but is not limited to: dihydroindolyl, isodihydroindolyl, dihydrobenzofuranyl, dihydrobenzothiophenyl, benzoxazolinonyl, etc. Exemplary 6-membered heterocyclyl fused with a C₆ aryl ring therein, also referred to as 6,6-bicycle heterocyclyl) includes, but is not limited to: tetrahydroquinolinyl, tetrahydroisoquinolyl, etc. A heterocyclyl group can be substituted with one or more substituents, for example, substituted with 1 to 5 substituents, 1 to 3 substituents, or 1 substituent.

The “C₆₋₁₀ aryl” refers to a group of a monocyclic or polycycle (such as bicycle) 4n+2 aromatic ring system having 6-10 cyclic carbon atoms and zero heteroatom (for example, having 6 or 10 π electrons shared in a cyclo arrangement) In some embodiments, the aryl has six cycle carbon atoms (“C₆ aryl”; such as phenyl) In some embodiments, the aryl has ten cyclic carbon atoms (“C₁₀ aryl”; such as naphtyl, such as 1-naphthyl and 2-naphthyl). The aryl further includes a ring system in which an aryl ring is fused with one or more cycloalkyl is or heterocyclyls, and a connection point is on the aryl ring; and in such case, the number of carbon atoms continuously represents the number of carbon atoms in the aryl ring system. An aryl group can be substituted with one or more substituents, for example, substituted with 1 to 5 substituents, 1 to 3 substituents, or 1 substituent

The “5- to 14-membered heteroaryl” refers to a group of a 5- to 14-membered monocyclic or bicyclic 4n+2 aromatic ring system having cyclic carbon atoms and 1-4 cyclic heteroatom (for example, having 6, 10, or 14 π electrons shared in a cyclic arrangement), where each heteroatom is independently selected from nitrogen, oxygen, and sulfur. In heteroaryl containing one or more nitrogen atoms, a connection point may be a carbon or nitrogen atom as long as the valence is acceptable. A heteroaryl bicyclic system may include one or more heteroatoms in one or two rings. The heteroaryl further includes a ring system in which a heteroaryl tog is fused with one or more cycloalkyls or heterocyclyls, and a connection point is on the heteroaryl ring; and in such case, the number of carbon atoms continuously represents the number of carbon atoms in the heteroaryl ring system. In some embodiments. 5- to 10-membered heteroaryl is preferred, which is a 5- to 10-membered monocyclic or bicycle 4n+2 aromatic ring system having cyclic carbon atoms and 1-4 cyclic heteroatoms. In other embodiments, 5- to 6-membered heteroaryl is particularly preferred, which is a 5- to 6-membered monocyclic or bicyclic 4n+2 aromatic ring system having cyclic carbon atoms and 1-4 cyclic heteroatoms. Exemplary 5-membered heteroaryl containing one heteroatom includes, but is not limited to: pyrroyl, furanyl, and thiophenyl. Exemplary 5-membered heteroaryl containing two heteroatoms includes, but is not limited to: imidazolyl, pyrazolyl, oxazolyl; isooxazolyl, thiazolyl, and isothiazolyl. Exemplary 5-membered heteroaryl containing three heteroatoms includes, but is not limited to: triazolyl, oxadiazolyl (such as 1,2,4-oxadiazolyl), and thiadiazolyl. Exemplary 5-membered heteroaryl containing four heteroatoms includes, but is not limited to: tetrazolyl. Exemplary 6-membered heteroaryl containing one heteroatom includes, but is not limited to: pyridyl. Exemplary 6-membered heteroaryl containing two heteroatoms includes, but is not limited to: pyridazinyl, pyrimidinyl, and pyrazoyl. Exemplary 6-membered heteroaryl containing three or four heteroatoms includes, but is not limited to: triazinyl and tetrazinyl, respectively. Exemplary 7-membered heteroaryl containing one heteroatom includes, but is not limited to: azepinyl, oxepanyl, and thiepinyl. Exemplary 5,6-bicyclic heteroaryl includes, but is not limited to: indolyl, isoindolyl, indazolyl, benzotriazolyl, benzothienyl, isobenzothienyl, benzofuranyl, benzoisofuranyl, benzimidazolyl, benzoxazolyl, benzoisoxazolyl, benzoxadiazolyl, benzothiazolyl, benzoisothiazolyl, benzothiadiazolyl, indolazinyl, and purinyl. Exemplary 6,6-bicyclic heteroaryl includes, but is not limited to: napthyridinyl, pteridinyl, quinolyl, isoquinolyl, cinnolinyl, quinozalinyl, phthalazinyl, and quinazolinyl. A heteroaryl group can be substituted with one or more substituents, for example, substituted with 1 to 5 substituents, 1 to 3 substituents, or 1 substituent.

The alkyl, the alkenyl, the alkynyl, the cycloalkyl, the heterocyclyl, the aryl, the heteroaryl and the bike defined herein are optional substituted groups.

Exemplary substituents on carbon atoms include, but are not limited to: halogen, —CN, —NO₂, —N₃, —SO₂H, —SO₃H, —OH, —OR^{aa}, —ON(R^{bb})₂, —N(R^{bb})₃⁺X⁻, —N(OR^{ee})R^{bb}, —SH, —SR^{aa}, —SSR^{ee}, —C(═)R^{aa}, —CO₂H, —CHO, —C(OR^{ee})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═)NR^{bb}SO₂R^{aa}, —NR^{bb}SO₂R^{aa}, —SO₂N(R^{bb})₂, —SO₂R^{aa}, —SO₂OR^{aa}, OSO₂R^{aa}, —S(═OR^{aa}, —OS(═O)R^{aa}, —Si(R^{aa})₃, —OSi(R^{aa}), —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)₂OR^{aa}, —SC(═O)R^{aa}, —P(═O)₂R^{aa}, —OP(═O)R^{aa}, —P(═O)(R^{aa})₂, —OP(═O)(R^{aa})₂, —OP(═O)(OR^{aa})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), alkyl, haloalkyl, alkenyl, alkynyl, cycloalkyl, heterocyclyl, aryl, and heteroaryl, where each of the alkyl, the alkenyl, the alkynyl, the cycloalkyl, the heterocyclyl, the aryl and the heteroaryl is independently substituted with 0, 1, 2, 3, 4, or 5 R^{dd} groups;
of two geminal hydrogens on carbon atoms are substituted with groups: ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{aa};
each R^{aa} is independently selected from alkyl, haloalkyl, alkenyl, alkynyl, cycloalkyl, heterocyclyl, aryl, and heteroaryl, or two R^{aa} groups are combined to form a heterocyclyl or heteroaryl ring, where each of the alkyl, the alkenyl, the alkynyl, the cycloalkyl, the heterocyclyl, the aryl and the heteroaryl is independently substituted with 0, 1, 2, 3, 4, or 5 Rad groups;
each R^{bb} is independently selected from hydrogen, —OH, —OR^{aa}, —N(R^{aa})₂, —CN, —C(═OR)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{aa})₂, —SO₂R^{cc}, —SO₂OR^{cc}, —SOR^{aa}, —C(═S)N(R^{cc}), —C(═O)SR^{cc}, —C(═S)SR^{cc}, —P(═O)(R^{aa})₂, —P(═O)(NR^{cc})₂, alkyl, haloalkyl, alkenyl, alkynyl, cycloalkyl, heterocyclyl, aryl, and heteroaryl, or two R^{bb} groups are combined to form a heterocyclyl or heteroaryl ring, where each of the alkyl, the alkenyl, the alkynyl, the cycloalkyl, the heterocyclyl, the aryl and the heteroaryl is independently substituted with 0, 1, 2, 3, 4, or 5 R^{dd} groups;
each R^{cc} is independently selected from hydrogen, alkyl, haloalkyl, alkenyl, alkynyl, cycloalkyl, heterocyclyl, aryl, and heteroaryl, or two R^{ee} groups are combined to form a heterocyclyl or heteroaryl ring, where each of the alkyl, the alkenyl, the alkynyl, the cycloalkyl, the heterocyclyl, the aryl and the heteroaryl is independently substituted with 0, 1, 2, 3, 4, or 5 R^{dd} groups;
each R^{dd} is independently selected from halogen, —CN, —NO₂, —N₃, —SO₂H, —SO₃H, —OH, —OR^{cc}, —ON(R^{ff})₂; —N(R^{ff})₂, —N(R^{ff})₃⁺X⁻; —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{aa}, —SO₂N(R^{ff})₂, —SO₂R^{ee}, —SO₂OR^{ee}, —OSO₂R^{ee}, —S(O)R^{ee}, —Si(R^{ee})₃, —OSi(R^{ee})₃, —(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(OR^{ee})₂, alkyl, haloalkyl, alkenyl, alkynyl, cycloalkyl, heterocyclyl, aryl, and heteroaryl, where each of the alkyl, the alkenyl, the alkynyl, the cycloalkyl, the heterocyclyl, the aryl and the heteroaryl is independently substituted with 0, 1, 2, 3, 4, or 5 R^{gg} groups, or two geminal R^{dd} substituents can be combined to form ═O or ═S;
each R^{ee} is independently selected from alkyl, haloalkyl, alkenyl, alkynyl, cycloalkyl, aryl, heterocyclyl, and heteroaryl, where each of the alkyl, the alkenyl, the alkynyl, the cycloalkyl, the heterocyclyl, the aryl and the heteroaryl is independently substituted with 0, 1, 2, 3, 4, or 5 R^{gg} groups;
each R^{ff} is independently selected from hydrogen, alkyl, haloalkyl, alkenyl, alkynyl, cycloalkyl, heterocycyl, aryl, and heteroaryl, or two R^{ff} groups are combined to form a heterocyclyl or heteroaryl ring, where each of the alkyl, the alkenyl, the alkynyl, the cycloalkyl, the heterocyclyl, the aryl and the heteroaryl is independently substituted with 0, 1, 2, 3, 4, or 5 R^{dd} groups;
each R^{gg} is independently: halogen, —CN, —NO₂, —N₃, —SO₂H, —SO₂H, —OH, —OCH₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻; —NH₃⁺X⁻, —N(OC₁₋₆, alkyl) (C₁₋₆ alkyl), —N(OH)(C₁₋₆ (alkyl), —NH(OH), —SH, —SC₁₋₆ alkyl, —SS(C₁₋₆ alkyl), —C(═O)(C₁₋₆ alkyl), —CO₃H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), NHCO₂(C₁₋₆ alkyl), —NHC(═O)N(C₁₋₆ alkyl)₂, —NHC(═O)NH(C₁₋₆ alkyl), —NHC(O)NH₂, —C(NH)O(C₁₋₆ alkyl), —OC(═NH) (C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO)₂(C₁₋₆ alkyl), —SO₂N(C₁₋₆ alkyl)₂, —SO₂NH(C₁₋₆ alkyl), —SO—NH₂; —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃, —C(═S)N(C₁₋₆ alkyl)₂, C(═S)NH(C₁₋₆ alkyl), C(═S)NH₂, C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ (alkyl)₂, C₁₋₆ alkyl, C₁₋₆ haloalkyl, C₂-C₆ alkenyl, C₃-C₇ alkynyl, C₃-C₇ cycloalkyl, C₆-C₁₀ aryl, C₃-C₇ heterocyclyl, or C₅-C₁₀ heteroaryl; or two geminal R^{gg} substituents can be combined to form ═O or ═S; where X⁻ is a counter ion.

Exemplary substituents on nitrogen atoms include, but are not limited to: hydrogen, —OH, —OR^{aa}, —N(R^{ee})₂; —CN, C(═O)R^{aa}, —C(═O)N(R^{aa})₂, —CO₂R^{aa}, —SO₂R^{aa}, —C(═NR^{bb})R^{aa}, —C(═NR^{aa})OR^{aa}, —C(═NR^{cc})N(R^{ee})₂, —SO₂N(R^{ee})₂, —SO₂R^{aa}, —SO₂OR^{aa}, —SOR^{aa}, —C(═S)N(R^{ee})₂, —C(═O)SR^{ee}, —C(S)SR^{ee}, —P(═O)₂R^{ee}, —P(═O)(R^{ee})₂, —P(═O)₂N(R^{ee})₂, —P(═O)(NR^{ee})₂, alkyl, haloalkyl, alkenyl), alkynyl, cycloalkyl, heterocyclyl, aryl, and heteroaryl, or two R^{ee} groups connected to nitrogen atoms are combined to form a heterocyclyl or heteroaryl ring, where each of the alkyl, the alkenyl, the alkynyl, the cycloalkyl, the heterocyclyl, the aryl and the heteroaryl is independently substituted with 0, 1, 2, 3, 4, or 5 R^{dd} groups, and R^{aa}, R^{bb}, R^{cc} and R^{dd} are described as above.

### Other Definition

The term “siRNA” herein is a double-stranded RNA molecule that can mediate silencing of a complementary target RNA (such as mRNA, such as a transcript of a gene that encodes a protein). The siRNA is usually double-stranded and includes an antisense strand complementary to the target RNA and a sense strand complementary to the antisense strand. For convenience, such mRNA is also called an mRNA to be silenced herein. Such gene is also called a target gene. Usually, an RNA to be silenced is an endogenous gene or a pathogen gene. In addition, an RNA except for the mRNA (such as a (RNA) and a viral RNA can also be targeted.

The term “antisense strand” refers to such a strand of the siRNA that includes a region that is completely, fully, or substantially complementary to a target sequence. The term “sense strand” refers to such a strand of the siRNA that includes a region that is completely, fully, or substantially complementary to the region of the term antisense strand defined herein.

The term “complementary region” refers to a region, on the antisense strand, that is completely, fully, or substantially complementary to a target mRNA sequence. In the case that the complementary region is not completely complementary to the target sequence, mispairing can be located in an internal or terminal region of a molecule. Usually, mispairing with highest tolerance is located in the terminal region, for example, within 5, 4, 3, 2, or 1 nucleotide at the 5′ and/or 3′ end. A part, most sensitive to mispairing, of the antisense strand is called a “seed region”. For example, in an siRNA including a 19nt strand, some mispairings can be tolerated at position 19 (from 5′ to 3).

The term “complementary” refers to the ability of a first polynucleotide to hybridize with a second polynucleotide under certain conditions such as strict conditions. For example, the strict conditions may include 400 mM NaCl, 40 mM PIPES with a pH value of 6.4, and 1 mM EDTA that are lasted at 50° C. or 70° C. for 12-16 hours. In order to meet the above requirements with respect to the hybridize ability, a “complementary” sequence may further include or completely form base pairs that are formed from non-Watson-Crick base pairs and/or from unnatural and modified nucleotides. Such non-Watson-Crick base pairs include, but are not limited to, G: U Wobble base pairs of Hoogstein base pairs.

A polynucleotide that is “at least partially complementary”, “fully complementary”, or “substantially complementary” to a messenger RNA (mRNA) refers to a polynucleotide that is substantially complementary to a continuous part of the mRNA of interest. For example, when a sequence is substantially complementary to a non-interrupted part of an mRNA that encodes PCSK9, a polynucleotide is at least partially complementary to the PCSK9 mRNA. Herein, the terms “complementary”, “completely complementary”, “fully complementary” and “substantially complementary” may be used relative to base pairs between the sense strand and the antisense strand of the siRNA, or between an antisense strand of an siRNA reagent and a target sequence.

The “fully complementarity” refers to an extent to which the sense strand merely needs to be complementary to the antisense strand in order to maintain overall double-stranded features of a molecule. In other words, although perfect complementarity is usually required, in some cases, especially in the antisense strand, one or more mispairings, such as 6, 5, 4, 3, 2, or) mispairing (relative to target mRNA) can be included, and the sense strand and the antisense strand can still maintain the overall double-stranded features of the molecule.

An “shRNA” refers to a short hairpin RNA. The shRNA includes two short inverted repeat sequences. The shRNA cloned into an shRNA expression vector includes two short inverted repeat sequences, which is separated by a loop sequence in the middle to form a hairpin structure and is controlled by a promoter polIII. Then, 5-6 T are linked to serve as a transcriptional terminator of an RNA polymerase III.

A “nucleoside” is a compound composed of two substances including a purine base or a pyrimidine base, and ribose or deoxyribose. A “nucleotide” is a compound composed of three substances including a purine base or a pyrimidine base, ribose or deoxyribose, and phosphoric acid. An “oligonucleotide” refers to, for example, a nucleic acid molecule (RNA or DNA) that has a length of less than 100, 200, 300, or 400 nucleotides

The “base” is a basic composition unit for synthesis of nucleosides, nucleotides, and nucleic acids, and due to nitrogen in composition elements, is also called “nitrogenous base”. Herein, unless otherwise specified, capital letters A, U, T, O and C represent base composition of nucleotides, which are adenine, uracil, thyme, guanine, and cytosine, respectively.

“Modification” of nucleotides described herein include, but are not limited to, methoxy modification, fluorinated modification, thiophosphate group ligation, or protection with conventional protective groups, etc. For example, the nucleotides with fluorinated modification refer to nucleotides formed by substitution of hydroxyl at site 2 of ribosyl of nucleotides with fluorine, and the nucleotides with methoxy modification refer to nucleotides formed by substitution of 2′-hydroxyl of ribosyl with methoxyl.

Herein, “modified nucleotides” include, but are not limited to, 2-O-methyl modified nucleotides, 2′-fluoro modified nucleotides, 2′-deoxy-modified nucleotides, inosine ribonucleotides, debasified nucleotides, reverse baseless deoxyribonucleotides, nucleotides containing a thiophosphate group, vinyl phosphate modified nucleotides, locked nucleotides, 2′-amino, modified nucleotides, 2′ alkyl-modified nucleotides, morpholino nucleotides, aminophosphates, unnatural bases containing nucleotides, and terminal nucleotides linked to cholesterol-based derivatives or a sebacamide dodecanoate group, deoxyribonucleotides, of nucleotides protected by conventional protective groups, etc. For example, the 2-fluoro modified nucleotides refer to nucleotides formed by substitution of hydroxyl at site 2′ of ribosyl of nucleotides with fluorine. The 2′-deoxy-modified nucleotides refer to nucleotides formed by substitution of 2 hydroxyl of ribosyl with methoxyl.

The “leaving group”, also known as a leave group, is an atom or a functional group that is detached from a larger molecule in a chemical reaction, which is a term used in a nucleophilic substitution reaction and an elimination reaction.

The “reactive phosphorus group” refers to a phosphorous-containing group contained in a nucleotide unit or a nucleotide analog unit, which can react with hydroxyl or amino contained in another molecule, especially another nucleotide unit or another nucleotide analog, through a nucleophile attack reaction. Usually, such reaction produces an ester internucleoside bond that links the first nucleotide unit of the first nucleotide analog unit to the second nucleotide unit or the second nucleotide analog unit. The reactive phosphorus group can be selected from phosphoramidite, H-phosphonate, alkyl-phosphonate, phosphate, or phosphate analogs, including but not limited to: natural phosphate, thiophosphate, dithiophosphate, borane phosphate, borane thiophosphate, phosphonate, halogen substituted phosphonate and phosphate, amino phosphate, phosphate diester, phosphate triester, thiophosphate diester, thiophosphate triester, diphosphate, and triphosphate, preferably —P(OCH₂CH₂CN)(N(iPr)₂).

The “protective group”, also known as a “protecting group”, refers to any atom or atom group that is added to a molecule to prevent an undesired chemical reaction of existing groups in the molecule. The “protective group” can be an unstable chemical component known in the field, which is used to protect reactive groups, such as hydroxyl, amino, and thiol group, to prevent an undesirable or inappropriate reaction during chemical synthesis. The protective group is usually selectively and/or orthogonally used to protect sites during reactions at other reactive sites, and can then be removed such that unprotected groups are left and kept intact or can be used m further reactions.

A non-restrictive list of the protective group includes benzyl; substituted benzyl; alkylcarbonyl and alkoxycarbonyl (such as tert-butoxycarbonyl (BOC), acetyl of isobutyryl); arylalkylcarbonyl and arylalkoxycarbonyl (such as benzyloxycarbonyl; substituted methyl ether (such as methoxymethyl ether), substituted ethyl ether; substituted benzyl ether; tetrahydropyranyl ether; silyl (such as trimethylsilyl, triisopropylsilyl, tert-butyldimethylsilyl, triisopropyl silyloxymethyl, 12-triethylsilyl, (trimethylsilyl)ethoxy]methyl, or tert-butyldiphenylsilyl); esters (such as benzoate), carbonates (such as methoxymethyl carbonate); sulfonates (such as tosylate or mesylate); acrylic ketal (such as dimethylacetal); cyclic ketal (such as 1,3-dioxane, 1,3-dioxolane, and those described herein), acyclic acetal; cyclic acetal (such as those described herein); acyclic hemiacetal, cyclic hemiacetal; cyclodithioketal (such as 1,3-dithiane or 1,3-dithiolane); orthoesters (such as those described herein), and triarylmethyl groups (such as triphenylmethyl, monomethoxy triphenylmethyl (MMTr): 4,4′-dimethoxytriphenylmethyl (DMTr), 4,4′,4″-trimethoxytriphenylmethyl (TMTr); and those described herein). The protective group is preferably selected from acetyl (Ac), benzoyl (Bzl), benzyl (Bn), isobutyryl (Bn), phenylacetyl, benzyloxy methylacetyl (BOM), β-methoxyethoxymethyl ether (MEM), methoxymethyl ether (MOM), p-methoxybenzyl ether (PMB), methylthiomethyl ether, pivaloyl (Piv), tetrahydropyranyl (THP), triphenylmethyl (Trt), methoxytriphenylmethyl[(4-methoxyphenyl) diphenylmethyl] (MMT), dimethoxytriphenylmethyl, [bis-(4-methoxyphenyl phenylmethyl (DMT), trimethylsilyl ether (TMS), tert-butyldimethylsilyl ether (TBDMS), tri-isopropylsilyloxymethyl ether (TOM), tri-isopropylsilyl ether (TIPS), methyl ether, ethoxyethyl ether (EE), N,N-dimethylformamidine, and 2-cyanoethyl (CE).

The “hydroxyl protective group” refers to a group that can prevent hydroxyl from a chemical reaction and can be removed under certain conditions to restore the hydroxyl. The hydroxyl protective group mainly includes a silane type protective group, an acyl type protective group, or an ether type protective group, and preferably includes the following groups:
trimethylsilyl (TMS), triethylsilyl) (TES), dimethylisopropylsilyl (DMIPS), diethylisopropylsdyl (DEIPS), tert-butyidimethylsilyl (TBDMS), tert-butyldiphenylsilyl (TBDPS), triisopropylsilyl (TIPS), acetyl (Ac), chloroacetyl, dichloroacetyl, trichloroacetyl, trifluoroacetyl (TFA), benzoyl, p-methoxybenzoyl, 9-fluorenylmethoxycarbonyl (Fmoc), allyloxycarbonyl (Alloc), 2,2,2-trichloroethoxycarbonyl (Troc), carbobenzoxy (Cbz), tert-butoxycarbonyl (Boc), phenylmethyl (Bn), p-methoxybenzyl (PMB), allyl, triphenylmethyl (Tr), di-p-methoxytriphenylmethyl (DMTr), methoxymethyl (MOM), phenoxymethyl (BOM), 2,2,2-trichloroethoxymethyl 2-methoxyethoxymethyl (MEM), methylthiomethyl (MTM), P-methoxybenzyloxymethyl (PMBM), —C(O)CH₂CH₂C(O)OH, or 4,4-dimethoxytriphenylmethyl.

The term “pharmaceutically acceptable salt” used herein refers to carboxylates or amino acid addition salts of the compound of the present invention, which are suitable for contact with tissues of patients without producing inappropriate toxicity, irritant effects, allergic reactions and the like within a reliable medical judgment range, are effective in their expected applications relative to a reasonable benefit/risk ratio, and include (if possible) a zwitterionic form of the compound of the present invention.

The present invention includes a tautomer, which is a functional group isomer produced by rapid movement of an atom at two positions in a molecule. A compound that exists in different tautomeric forms is not limited to any particular tautomers, but is intended to cover all of the tautomeric forms.

The compound of the present invention may include one or more asymmetric centers, and thus may exist in a variety of stereoisomer forms, such as an enantiomer form and/or a diastereoisomer form. For example, the compound of the present invention may be a separate enantiomer, a diastereoisomer or a geometric isomer (such as a cis-isomer and a trans-isomer), or may be in the form of a mixture of stereoisomers, including a racemate mixture and a mixture containing one or more stereoisomers. Isomers can be separated from a mixture by a method known to persons skilled in the art. The method includes: chiral high-pressure liquid chromatography (HPLC) and formation and crystallization of a chiral salt; and alternatively, preferred isomers can be prepared by asymmetric synthesis.

The present invention further includes isotopically labeled compounds (isotopic variants) that are equivalent to the one of Formula (I), but one or more atoms are substituted with atoms with atomic masses or mass numbers different from atomic masses or mass numbers commonly found in nature. Examples of isotopes that can be introduced into the compound of the present invention include isotopes of hydrogen, carbon, nitrogen, oxygen, phosphorus, sulfur, fluorine, and chlorine, such as ²H, ³H, ¹³C, ¹³C, ¹⁴C, ¹⁵N, ¹⁸O, ¹⁷O, ³¹P, ³²P, ³⁵S, ¹⁸F, and ³⁶Cl, respectively. All compounds of the present invention containing the isotopes and/or other isotopes of other atoms, precursor drugs thereof, and pharmaceutically acceptable salts of the compounds or the precursor drugs fall within the scope of the present invention. Certain isotopically labeled compounds of the present invention, such as those into which radioisotopes (such as ³H and ¹⁴C) are introduced, can be used for determining distribution of drugs and/or substrate tissues. Tritium, namely ³H, and carbon-14, namely ¹⁴C isotope, are particularly preferred because they are easy to prepare and detect. Furthermore, substitution with heavier isotopes, such as deuterio, namely ²H, may be preferred in some cases because higher metabolic stability can provide therapeutic benefits, such as prolonging the half-life in vivo or reducing dosage requirements. Isotopically labeled compounds of Formula (I) of the present invention and precursor drugs thereof can generally be prepared by substituting non-isotopically labeled reagents with readily available isotopically labeled reagents in processes described below and/or technologies disclosed in examples and preparative examples.

### Compound of the Present Invention

The present invention specifically relates to a nucleotide dimer as shown in Formula (A)

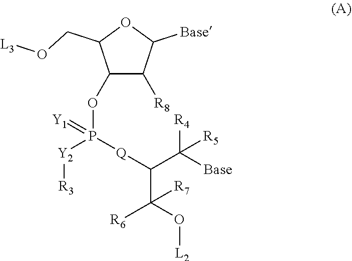

where various groups are as defined in the context.
Q

In one embodiment, the Q is —X—; and in another embodiment, the Q is —X—O—.
X

In one embodiment, the X is a chemical bond, in another embodiment, the X is —(CR₁R₂)ₘ—; and in another embodiment, the X is —CR₁═CR₂—.
Y₁ and Y₂

In one embodiment, the Y₁ is O; in another embodiment, the Y₁ is S; and in another embodiment, the Y₁ is NR.

In one embodiment, the Y₂ is O; in another embodiment, the Y₂ is S; and in another embodiment, the Y₂ is a chemical bond.
L₂ and L₃

In one embodiment, the L₂, is H; and in another embodiment, the L₂ is P₂.

In one embodiment, the L₃ is H; and in another embodiment, the L₂ is P₃.

R₁, R₂, R₄, R₅, R₆ and R₇

In one embodiment, the R₁ is H; in another embodiment, the R₁ is D; in another embodiment, the R₁ is halogen; in another embodiment, the R₁ is OH; in another embodiment, the R₁ is CN; in another embodiment, the R₁ is C₁₋₆ alkyl, such as C₁₋₆ alkyl; in another embodiment, the R₁ is C₁₋₆ haloalkyl, such as C₁₋₄ haloalkyl; in another embodiment, the R₁ is C₂₋₆ alkenyl, and in another embodiment, the R₁ is C₂₋₆ alkynyl.

In one embodiment, the R₂ is H; in another embodiment, the R₂ is D; in another embodiment, the R₂ is halogen; in another embodiment, the R₂ is OH; in another embodiment, the R₂ is CN; in another embodiment, the R₂ is C₁₋₆ alkyl, such as C₁₋₄ alkyl; in another embodiment, the R₂ is C₁₋₆ haloalkyl, such as C₁₋₆ haloalkyl; in another embodiment, the R₂ is C₂₋₆ alkenyl; and in another embodiment, the R₂ is C₂₋₆ alkynyl.

In one embodiment, the R₂ is H; in another embodiment, the R₂ is D; in another embodiment, the R₂ is halogen; in another embodiment, the R₄ is OH; in another embodiment, the R₂ is CN; in another embodiment, the R₂ is C₁₋₆ alkyl, such as C₁₋₄ alkyl; in another embodiment, the R₂ is C₃₋₆ haloalkyl, such as C₁₋₄ haloalkyl; in another embodiment, the R₂ is C₂₋₆ alkenyl; and in another embodiment, the R₂ is C₂₋₆ alkynyl.

In one embodiment, the R₅ is H; in another embodiment, the R₅ is D; in another embodiment, the R₅ is halogen, in another embodiment, the R₅ is OH; in another embodiment, the R₅ is CN, in another embodiment, the R₅ is C₁₋₆ alkyl, such as C₁₋₄ alkyl; in another embodiment, the R₅ is C₁₋₆ haloalkyl, such as C₁₋₄ haloalkyl; in another embodiment, the R₅ is C₂₋₆ alkenyl; and in another embodiment, the R₅ is C₂₋₆ alkynyl.

In one embodiment, the R₆ is H; in another embodiment, the R₅ is D; in another embodiment, the R₆ is halogen; in another embodiment, the R₆ is OH; in another embodiment, the R₆ is CN; in another embodiment, the R₆ is C₁₋₆ alkyl, such as C₁₋₄ alkyl, in another embodiment, the R₆ is C₁₋₆ haloalkyl, such as C₁₋₄ haloalkyl, in another embodiment, the R₆ is C₂₋₆ alkenyl; and in another embodiment, the R₆ is C₂₋₆ alkynyl.

In one embodiment, the R₇ is H; in another embodiment, the R₇ is D; in another embodiment, the R₇ is halogen; in another embodiment, the R₇ is OH; in another embodiment, the R₇ is CN, in another embodiment, the R₇ is C₁₋₆ alkyl, such as C₁₋₄ alkyl; in another embodiment, the R₁ is C₁₋₆ haloalkyl, such as C₁₋₄ haloalkyl; in another embodiment, the R₇ is C₂₋₆ alkenyl; and in another embodiment, the R₇ is C₂₋₆ alkynyl.

In one embodiment, the R₁, the R₂, the R₄, the R₅, the R₆ and the R₇ are each independently and optionally unsubstituted; and in another embodiment, the R₁, the R₂, the R₄, the R₅, the R₆ and the R₇ are each independently and optionally substituted with 1, 2, 3, 4, 5, 6, 7, 8, or more R′(s).
R₃

In one embodiment, the R₃ is H; in another embodiment, the R₃ is C₁₋₆ alkyl, preferably C₁₋₄ alkyl; in another embodiment, the R₂ is C₁₋₆ cyanoalkyl; in another embodiment, the R₂ is C₁₋₄ cyanoalkyl, such as cyanoethyl; in another embodiment, the R₃ is C₂₋₆ haloalkyl, preferably C₁₋₄ haloalkyl; in another embodiment, the R₃ is C₂₋₆ alkenyl, in another embodiment, the R₅ is C₂₋₆ alkynyl, in another embodiment, the R₅ is C₃₋₁₆ cycloalkyl; in another embodiment, the R₂ is 3- to 10-membered heterocyclyl; in another embodiment, the R₃ is C₆₋₁₀ aryl; and in another embodiment, the R₃ is 5- to 14-membered heteroaryl.

In one embodiment, the R₃ is unsubstituted; and in another embodiment, the R₃ is optionally substituted with 1, 2, 3, 4, 5, 6, 7, 8, or more R′(s).
R₈

In one embodiment, the R₈ is H; in another embodiment, the R₈ is D; in another embodiment, the R₈ is OH; in another embodiment, the R₈ is halogen, such as fluorine; in another embodiment, the R₈ is C₁₋₆ alkyl, preferably C₁₋₄ alkyl; in another embodiment, the R₈ is C₁₋₆ haloalkyl, preferably C₁₋₄ haloalkyl; and in another embodiment, the R₈ is C₁₋₆ alkoxyl, preferably C₁₋₄ alkoxyl, such as methoxyl.
Base and Base′

In one embodiment, the Base is H; in another embodiment, the Base is a modified or unmodified base or a leaving group; and in another embodiment, the Base is selected from modified or unmodified A, U, T, G, and C, such as

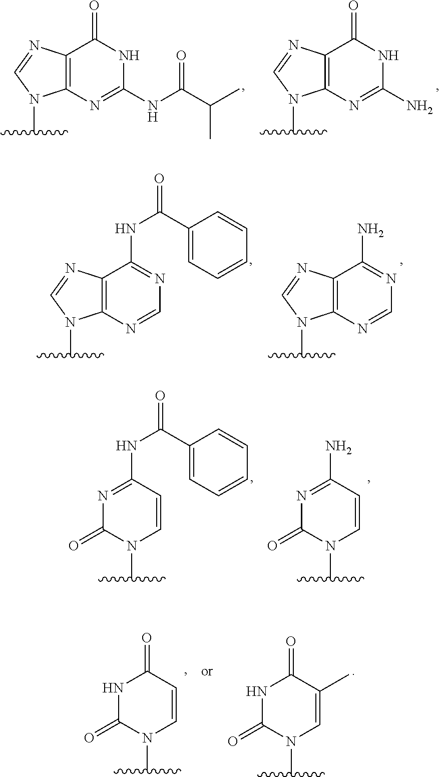

In one embodiment, the Base′ is H; in another embodiment, the Base′ is a modified or unmodified base or a leaving group, and in another embodiment, the Base′ is selected from modified or unmodified A, U, T, G, and C, such as

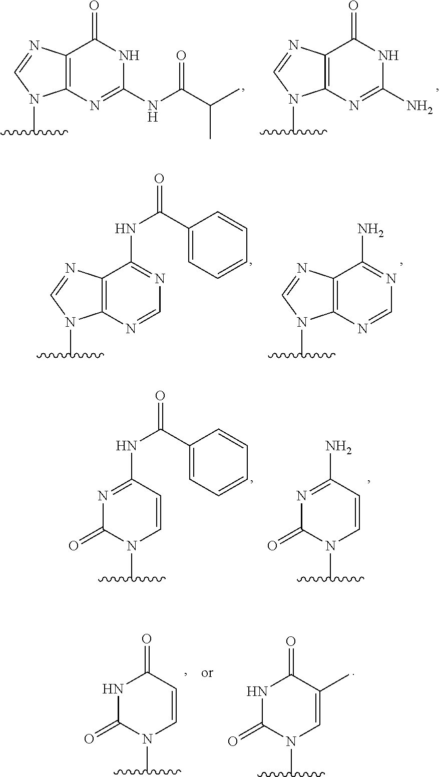

P₂ and P₃

In one embodiment, the P₂ is a hydroxyl protective group; and in another embodiment, the P₂ is a reactive phosphorus group, such as —P(OCH₂CH₂CN)(N(iPr)₂).

In one embodiment, the P₃ is a hydroxyl protective group, preferably a silane protective group, an acyl protective group, or an ether protective group, such as DMTr.
R and R′

In one embodiment, the R is H; in another embodiment, the R is C₁₋₆ alkyl, such as C₁₋₄ alkyl; and in another embodiment, the R is C₁₋₆ haloalkyl.

In one embodiment, the R′ is D; in another embodiment, the R′ is halogen; in another embodiment, the R′ is CN; in another embodiment, the R′ is C₁₋₆ alkyl; in another embodiment, the R′ is C₁₋₆ haloalkyl; in another embodiment, the R′ is C₂₋₆ alkenyl; in another embodiment, the R′ is C₂₋₆ alkynyl; in another embodiment, the R′ is C₃₋₁₀ cycloalkyl; in another embodiment, the R′ is 3- to 10-membered heterocyclyl; in another embodiment, the R′ is C₆₋₁₉ aryl; in another embodiment, the R′ is 5- to 14-membered heteroaryl; and in another embodiment, the R′ is selected from —OR^{a}, —OC(O)R^{a}, —C(O)R^{a}, —C(O)OR^{a}, —C(O)NR^{a}R^{b}, —S(O)ₙR^{a}, —S(O)ₙOR^{a}, —S(O)ⁿNR^{a}R^{b}, —NR^{a}R^{b}, —NR^{a}C(O)R^{b}, —NR^{a}—C(O)OR^{b}, —NR^{a}—S(O)ₙR^{b}, or —NR^{a}C(O)NR^{a}R^{b}.

R^{a} and R^{b} are independently selected from H, C₁₋₆ alkyl, C₂₋₆ alkenyl, C₂₋₆ alkynyl, C₃₋₁₀ cycloalkyl, 3- to 10-membered heterocyclyl, C₆₋₁₀ aryl, or 5- to 14-membered heteroaryl; or the R^{a} and the R^{b} as well as nitrogen atoms connected thereto form 3- to 10-membered heterocyclyl.
n is independently selected from 1 or 2.
m

In one embodiment, the m is 1; in another embodiment, the m is 2; in another embodiment, the m is 3; m another embodiment, the m is 4; and in another embodiment, the m is 5
GalNAc

In one embodiment, the GalNAc is a conjugate group as shown in Formula (X):

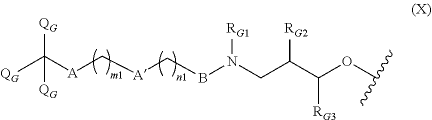

where,
[symbol] represents a position connected with a biomolecule;
Q_{G1} is independently H,

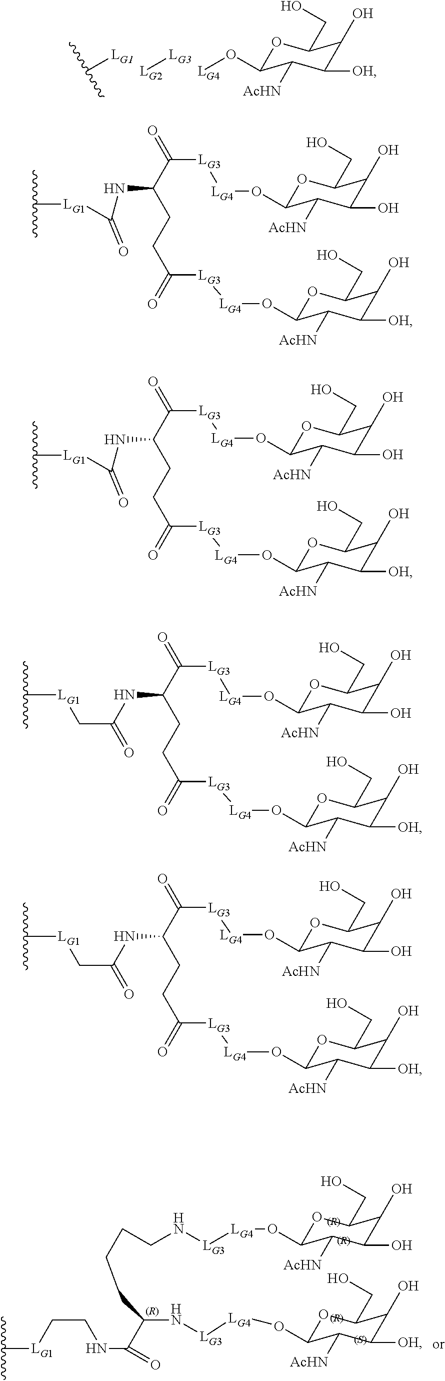

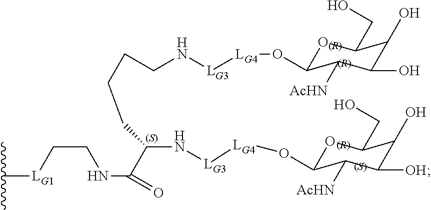

L_{G1} is a chemical bond, —CH₂, —CH₂CH₂, —C(O)—, —CH₂O—, —CH₂O—CH₂CH₂O—, or —NHC(O)—(CH₂NHC(O)ₙ—;
L_{G2} is a chemical bond or —CH₂CH₂C(O)—;
L_{G3} is a chemical bond, —(NHCH₂CH₂)_{b}—, (NHCH₂CH—CH₂)—, or —C(O)CH₂—;
L_{G4} is —(OCH₂CH₂)_{c}—, —(OCH₂CH₂CH₂)_{c}—, —(OCH₂CH₂CH₂CH₂)_{c}—, (OCH₂CH₂CH₂CH₂CH₂)_{c}—, or —NHC(O)—(CH₂)₄—;
where a is 0, 1, 2, or 3;
b is 1, 2, 3, 4, or 5;
c is 1, 2, 3, 4, or 5;
d is 1, 2, 3, 4, 5, 6, 7, or 8;
A is a chemical bond, —CH₂O—, or —NHC(O)—;
A′ is a chemical bond, —C(O)NH—, —NHC(O)—, or —(CH₂CH₂O)_{c}—;
where e is 1, 2, 3, 4, or 5;
B is a chemical bond, —CH₂, —C(O)—, -M-, —CH₂-M-, or —C(O)-M-;
where M is

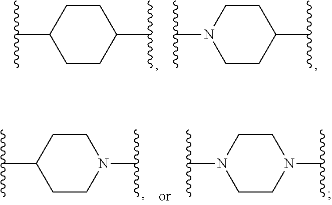

R_{G1} and R_{G2} together form —CH₂CH₂O— or —CH₂CH(R_{G})—O—, and R_{G3} is H;
or the R_{G1} and the R_{G3} together form —C₁₋₂ alkylene-, and the R_{G2} is H;
where R_{G} is —OR_{G}′, —CH₂OR_{G}′, or —CH—CH₂OR_{G}′, where R_{G}′ is H, a hydroxyl protective group or a solid phase carrier, and the hydroxyl protective group is preferably —C(O)CH₂CH₂C(O)OH or 4,4′-dimethoxytriphenylmethyl;
m1 is 0, 1, 2, 3, 4, 5, 6, 7, 8, 9, or 10; and
n1 is 0, 1, 2, 3, 4, 5, 6, 7, 8, 9, or 10.

In another embodiment, the GalNAc is a conjugate group as shown in Formula (I′):

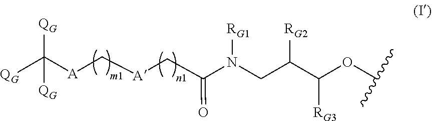

where,
[symbol] represents a position connected with a biomolecule;
Q_{G} is independently H,

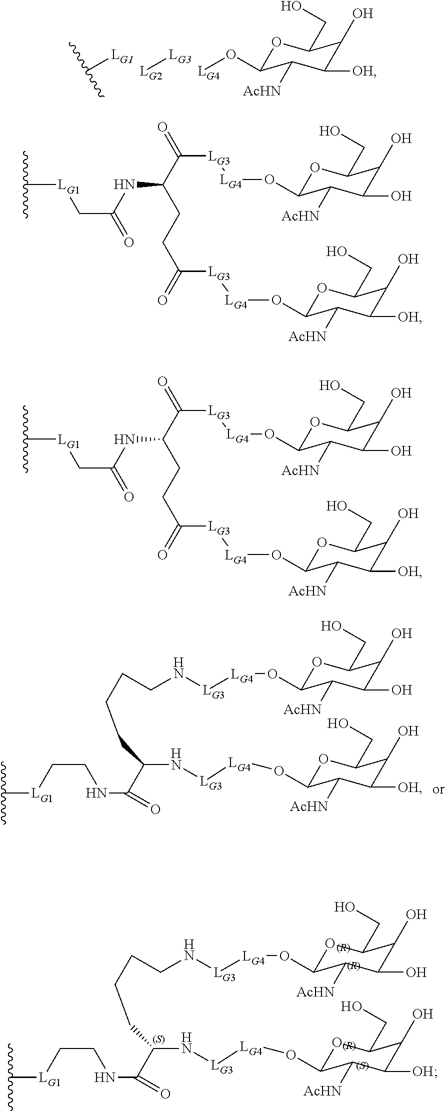

L_{G1} is a chemical bond, —CH₂—, —CH₂CH₂—, —C(O)—, —CH₂O—, —CH₂O—CH₂CH₂O—, or —NHC(O)—(CH₂NHC(O))_{c}—;
L_{G2} is a chemical bond or —CH₂CH₂C(O)—;
L_{G3} is a chemical bond, —(NHCH₂CH₂)_{b}—, —(NHCH₂CH₂CH₂)_{b}—, or —C(O)CH₂;
L_{G4} is —(OCH₂CH₂)_{c}—, —(OCH₂CH₂CH₂)_{c}—, —(OCH₂CH—CH₂CH₂)_{c}—, —(OCH₂CH₂CH₂CH₂CH₂)_{c}—, or —
where a is 0, 1, 2, or 3;
b is 1, 2, 3, 4, or 5;
c is 1, 2, 3, 4, or 5;
d is 1, 2, 3, 4, 5, 6, 7, or 8;
A is —CH₂O— or —NHC(O)—;
A′ is a chemical bond, —C(O)NH—, or —NHC(O)—;
R_{G1} and R_{G2} together form —CH—CH₂O— or —CH₂CH(R_{G})—O—, and R_{G3} is H;
or the R_{G1} and the R_{G3} together form —C₁₋₂ alkylene-, and the R_{G2} is H;
where R_{G} is —OR_{G}′, —CH₂OR_{G}′, or —CH₂CH₂OR_{G}′, where R_{G}′ is H, a hydroxyl protective group or a solid phase carrier, and the hydroxyl protective group is preferably —C(O)CH₂CH₂C(O)OH or 4,4′-dimethoxytriphenylmethyl;
m1 is 0, 1, 2, 3, 4, 5, 6, 7, 8, 9, or 10; and
n1 is 0, 1, 2, 3, 4, 5, 6, 7, 8, 9, or 10.

In another embodiment, the GalNAc is a conjugate group as shown in Formula (X), where,
the Q_{G} is independently H,

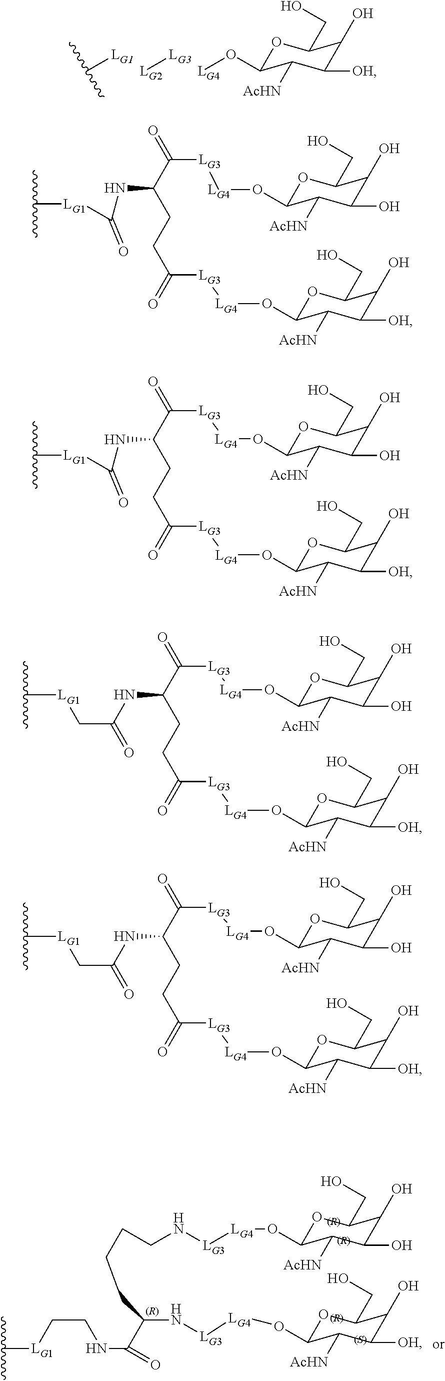

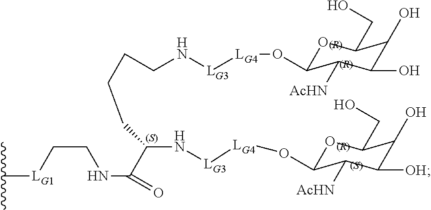

the L_{G1} is a chemical bond, —CH₂—, —CH₂CH₂—, —C(O)—, —CH₂O—, —CH₂O—CH₂CH₂O—, or —NHC(O)—(CH₂NHC(O))ₐ—;
the L_{G2} is a chemical bond or —CH—CH₂C(O)—;
the L_{G3} is a chemical bond, —(NHCH₂CH₂)—, —(NHCH₂CH₂CH₂)—, or —C(O)CH₂;
the L_{G4} is —(OCH₂CH₂)_{c}—, —(OCH₂CH₂CH₂)_{c}—, —(OCH₂CH₂CH₂CH₂)_{c}—, —(OCH₂CH₂CH₂CH₂CH₂)_{c}—, or —NHC(O)—(CH₂)_{d}—;
where the a is 0, 1, 2, or 3;
the b is 1, 2, 3, 4, or 5;
the c is 1, 2, 3, 4, or 5,
the d is 1, 2, 3, 4, 5, 6, 7, or 8;
the A is a chemical bond, —CH₂O—, or —NHC(O)—;
the A′ is a chemical bond, —C(O)NH—, —NHC(O)—, or —((CH₂CH₂O)ₙ—;
where the e is 1, 2, 3, 4, or 5;
the B is a chemical bond, —CH₂—, -M-, —CH₂-M-, or —C(O)-M-;
where the M is

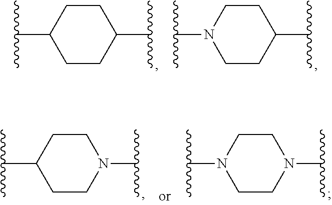

the R_{G1} and the R_{G2} together form —CH₂CH₂O— or —CH₂CH(R_{G})—O—, and the R_{G3} is H;
or the R_{G1} and the R_{G3} together form —C₁₋₂ alkylene-, and the R_{G2} is H;
where the R_{G} is —OR_{G}′, —CH₂OR_{G}′, or —CH₂CH₂OR_{G}′, where the R_{G}′ is H, a hydroxyl protective group or a solid phase carrier, and the hydroxyl protective group is preferably —C(O)CH₂CH₂C(O) OH or 4,4′-dimethoxytriphenylmethyl;
the m1 is 0, 1, 2, 3, 4, 5, 6, 7, 8, 9, or 10; and
the n1 is 0, 1, 2, 3, 4, 5, 6, 7, 8, 9, or 10.

In another embodiment, the GalNAc is a conjugate group as shown in Formula (X), where,
the Q_{G} is independently H,

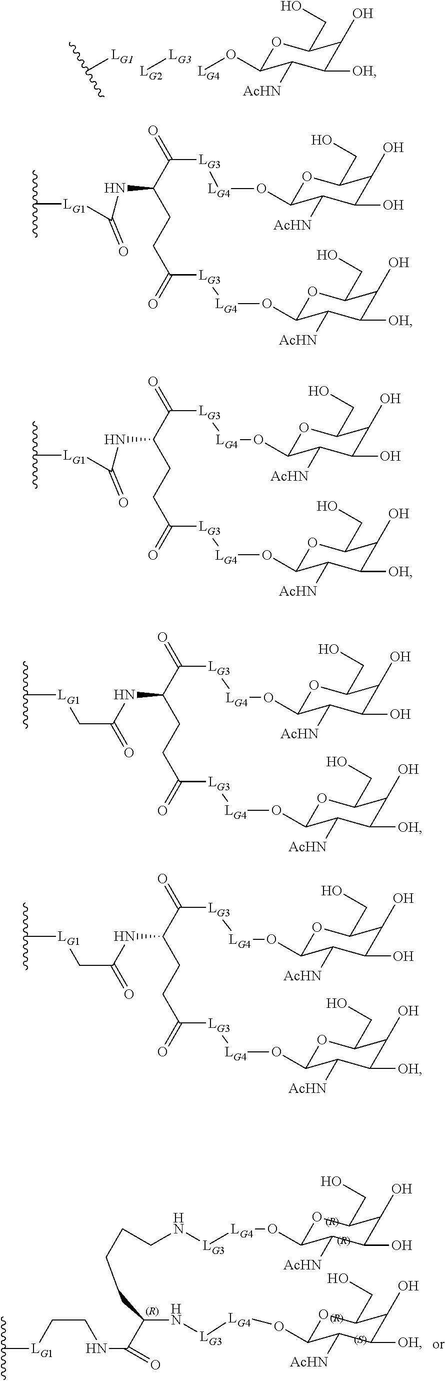

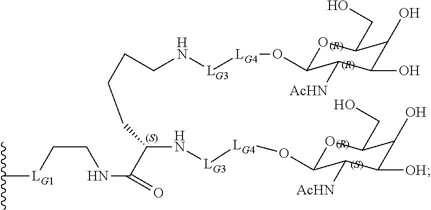

the L_{G1} is a chemical bond, —CH₂, —CH₂CH₂, —C(O)—, —CH₂O—, —CH₂O—CH₂CH₂O—, or —NHC(O)—(CH₂NHC(O))ₙ—;
the L_{G2} is a chemical bond or —CH₂CH₂C(O)—;
the L_{G3} is a chemical bond, —(NHCH₂CH₂)—, —(NHCH₂CH₂CH₂)_{b}—, or —C(O)CH₂—;
the L_{G4} is —(OCH₂CH₂)_{c}—, —(OCH₂CH₂CH₂)_{c}—, (OCH₂CH—CH₂CH₂)_{c}—, —OCH₂CH₂CH₂CH—CH₂)_{c}—, or —NHC(O)—(CH₂)_{d}—;
where the a is 0, 1, 2, or 3;
the b is 1, 2, 3, 4, or 5;
the c is 1, 2, 3, 4, or 5;
the d is 1, 2, 3, 4, 5, 6, 7, or 8;
the A is a chemical bond, —CH₂O—, or —NHC(O)—;
the A′ is —OCH₂CH₂O);
where the e is 1, 2, 3, 4, or 5;
the B is a chemical bond, —CH₂—, —C(O)—, -M-, —CH₂-M-, or —C(O)-M;
where the M is

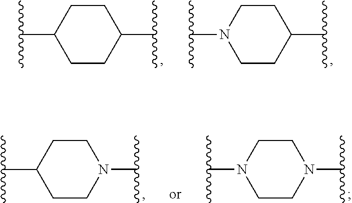

the R_{G1} and the R_{G2} together form —CH₂CH₂O— or —CH—CH(R_{G})—O—, and the Ring is H;
or the R_{G1} and the R_{G3} together form —C₁₋₂ alkylene-, and the R_{G2} is H;
where the R_{G} is —OR_{G}′, —CH₂OR_{G}′, or —CH₂CH₂OR_{G}′, where the R_{G}′ is H, a hydroxyl protective group of a solid phase carrier, and the hydroxyl protective group is preferably —C(O)CH₂CH₂C(O) OH or 4,4′-dimethoxytriphenylmethyl;
the m1 is 0, 1, 2, 3, 4, 5, 6, 7, 8, 9, or 10; and
the n1 is 0, 1, 2, 3, 4, 5, 6, 7, 8, 9, or 10.

Any one of technical solutions or combinations thereof in any one of the above specific embodiments can be combined with any one of technical solutions or any combinations thereof in other specific embodiments. For example, any one of technical solutions or any combinations thereof of the Q can be combined with any one of technical solutions or any combinations thereof of the X, the Y₁, the Y₂, the L₂, the L₂, the R₁, the R₂, the R₃, the R₄, the R₅, the R₆, the R₇, the R₈, the Base, and the Base′, etc. The present invention is intended to include combinations of all of these technical solutions, which are not listed in detail due to a limited space.

In one aspect, the present invention specifically relates to the following technical solutions:
A1. A nucleotide monomer as shown in Formula I,

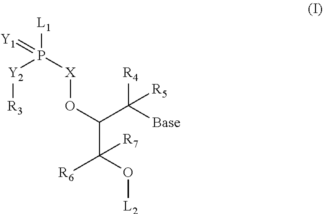

where,
L₁ is OH, OP₁, or a chemical bond of hydroxyl at the 2′ or 3′ end of ribose that is connected to another nucleotide, nucleoside, or oligonucleotide;
L₂ is H, P₂, or a chemical bond of a phosphate P atom at the 5′ end of ribose that is connected to another nucleotide, nucleoside, or oligonucleotide;
X is a chemical bond or CR₁R₂;
Y₁ is O, S, or NR;
Y₂ is O, S, or a chemical bond;
R₁, R₂, R₄, R₅, R₆ and R₇ are independently selected from H, D, halogen, CN, C₁₋₆ alkyl. C₁₋₆ haloalkyl, C₂₋₆ alkenyl, C₂₋₆ alkynyl, C₃₋₁₀ cycloalkyl, 3- to 10-membered heterocyclyl, C₆₋₁₀ aryl, or 5- to 14-membered heteroaryl, and are optionally substituted with 1, 2, 3, 4, 5, 6, 7, 8 or more R′(s);
R₃ is selected from H, C₁₋₆ alkyl, C₁₋₆ haloalkyl, C₂₋₆ alkenyl, C₂₋₆ alkynyl, C₃₋₁₀ cycloalkyl, 3- to 10-membered heterocyclyl, C₆₋₁₀ aryl, or 5- to 14-membered heteroaryl, and is optionally substituted with 1, 2, 3, 4, 5, 6, 7, 8 or more R′(s);
Base is H, a modified or unmodified base, or a leaving group,
P₁ and P₂ are independently selected from a hydroxyl protective group, preferably a silane protective group, an acyl protective group, or an ether protective group, more preferably
trimethylsilyl (TMS), triethylsilyl (TES), dimethylisopropylallyl (DMIPS), diethylisopropylsilyl (DEIPS), tert-butyidimethylsilyl (TBDMS), tert-butyldiphenylsilyl (TBDPS), triisopropylsilyl (TIPS).
acetyl (Ac), chloroacetyl, dichloroacetyl, trichloroacetyl, trifluoroacetyl (TFA), benzoyl, p-methoxybenzoyl, 9-fluorenylmethoxycarbonyl (Fmoc), allyloxycarbonyl (Alloc), 2,2,2-trichloroethoxycarbonyl (Troc), carbobenzoxy (Cbz), tert-butoxycarbonyl (Boc),
phenylmethyl (Bn), p-methoxybenzyl (PMB), allyl, triphenylmethyl (Tr), di-p-methoxytriphenylmethyl (DMTr), methoxymethyl (MOM), phenoxymethyl (BOM), 2,2,2-trichloroethoxymethyl, 2-methoxyethoxymethyl (MEM), methylthiomethyl (MTM), or p-methoxybenzyloxymethyl (PMBM);
R is selected from H, C₁₋₆ alkyl, or C₁₋₆ haloalkyl;
R′ is selected from D, halogen, CN, C₁₋₆ alkyl, C₁₋₆ haloalkyl, C₂₋₆ alkenyl, C₂₋₆ alkynyl, C₃₋₁₀ cycloalkyl, 3- to 10-membered heterocyclyl, C₆₋₁₀ aryl, 5- to 14-membered heteroaryl, —OR^{a}, —OC(O)R^{a}, —C(O)R^{a}, —C(O)OR^{a}, —C(O)NR^{a}R^{b}, —S(O)ₙR^{a}, —S(O)ₙOR^{a}, —S(O)ₙNR^{a}R^{b}, —NR^{a}R^{b}, —NR^{a}C(O)R^{b}, —NR^{a}—C(O)OR^{b}, —NR^{a}—S(O)ₙR^{b}, or —NR^{a}C(O)NR^{a}R^{b};
n is independently 1 or 2; and
R^{a} and R^{b} are independently selected from H, C₁₋₆ alkyl, C₂₋₆ alkenyl, C₂₋₆ alkynyl, C₃₋₁₀ cycloalkyl, 3- to 10-membered heterocyclyl, C₆₋₁₀ aryl, or 5- to 14-membered heteroaryl; or the R^{a} and the R^{b} as well as nitrogen atoms connected thereto form 3- to 10-membered heterocyclyl.
A2. The nucleotide monomer according to the technical solution A1, where the Lt is a chemical bond of hydroxyl at the 2 or 3 end of ribose that is connected to another nucleotide, nucleoside, or oligonucleotide, and the L; is preferably a chemical bond of hydroxyl at the 3′ end of ribose that is connected to another nucleotide, nucleoside, or oligonucleotide.
A3. The nucleotide monomer according to the technical solution A1 or A2, where the L₂ is H, or a chemical bond of a phosphate P atom at the 5′ end of ribose that is connected to another nucleotide, nucleoside, or oligonucleotide; the L₂ is preferably H; and the L₂ is preferably a chemical bond of a phosphate P atom at the 5′ end of ribose that is connected to another nucleotide, nucleoside, or oligonucleotide.
A4. The nucleotide monomer according to any one of the technical solutions A1-A3, where the X is a chemical bond.
A5. The nucleotide monomer according to any one of the technical solutions A1-A3, where the X is CR₁R₂, preferably CH₂.
A6. The nucleotide monomer according to any one of the technical solutions A1-A5, where the Y₁ is O.
A7. The nucleotide monomer according to any one of the technical solutions A1-A6, where the Y₂ is O.
A8. The nucleotide monomer according to any one of the technical solutions A1-A7, where the R₁, the R₂, the R₄, the R₅, the R₆ and the R₇ are independently selected from H, D, halogen, CN, C₁₋₆ alkyl, C₁₋₆ haloalkyl, C₂₋₆ alkenyl, C₂₋₆ alkynyl, C₄₋₈ cycloalkyl, 4- to 7-membered heterocyclyl, C₆₋₁₀ aryl, and 5- to 6-membered heteroaryl, preferably H, D, halogen, CN, C₁₋₆ alkyl, C₁₋₆ haloalkyl, C₂₋₆ alkenyl, or C₂₋₆ alkynyl, preferably H, D, halogen, or C₁₋₆ alkyl, and more preferably H or D
A9. The nucleotide monomer according to any one of the technical solutions A1-A5, where the R₃ is selected from H. C₁₋₆ alkyl, C₁₋₆ haloalkyl, C₂₋₆ alkenyl, C₂₋₆ alkynyl, C₃₋₁₀ cycloalkyl, 3- to 10-membered heterocyclyl, C₆₋₁₀ aryl, or 5- to 14-membered heteroaryl, preferably H, C₁₋₆ alkyl, or C₁₋₆ haloalkyl.
A10. The nucleotide monomer according to any one of the technical solutions A1-A9, where the Base is a modified or unmodified base, preferably modified or unmodified A, U, T, G, and C, and more preferably modified or unmodified G or C.
A11. The nucleotide monomer according to any one of the technical solutions A1-A9, where the Base is a leaving group that is preferably selected from halogen, hydroxyl, —OCOR″, —OTs, or —ONO₂, where R″ is C₁₋₆ alkyl, C₂₋₆ alkenyl, C₂₋₆ alkynyl, C₆₋₁₀ cycloalkyl, 3- to 10-membered heterocyclyl, C₆₋₁₀ aryl, or 5- to 14-membered heteroaryl, preferably, the Base is halogen or —OTs, and more preferably, the Base is —OTs.
A12. The nucleotide monomer according to the technical solution A1, having one of the following structures:

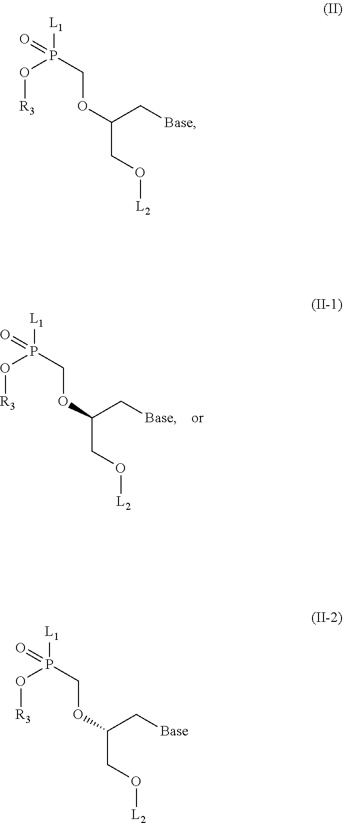

where,
L₁ is OH, OPt, or a chemical bond of hydroxyl at the 3′ end of ribose that is connected to another nucleotide, nucleoside, or oligonucleotide;
L₂ is H, P₂, or a chemical bond of a phosphate P atom at the 5′ end of ribose that is connected to another nucleotide, nucleoside, or oligonucleotide;
R₃ is selected from H, C₁₋₆ alkyl, or C₁₋₆ haloalkyl; and
Base is a modified or unmodified base, preferably modified or unmodified A, U, T. G, and C, and more preferably modified or unmodified G or C.
A13. A nucleotide dimer as shown in Formula III,

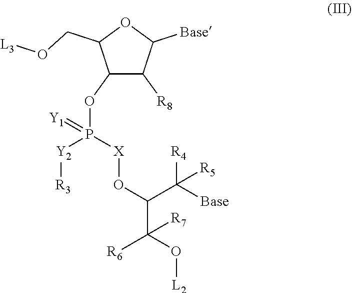

where,
L₃ is H, P₂ or a chemical bond of a phosphate P atom at the 2′ or 3′ end of ribose that is connected to another nucleotide, nucleoside, or oligonucleotide;
R₈ is selected from H, OH, halogen, C₁₋₆ alkyl, C₁₋₆ haloalkyl, or C₁₋₆ alkoxyl, preferably fluorine or methoxyl, and preferably H;
Base′ is H, a modified or unmodified base, or a leaving group, preferably modified or unmodified A, U, T, G, and C; and
other groups are as defined according to any one of the technical solutions A1-A12.
A14. The nucleotide dimer according to the technical solution A13, having the following structure:

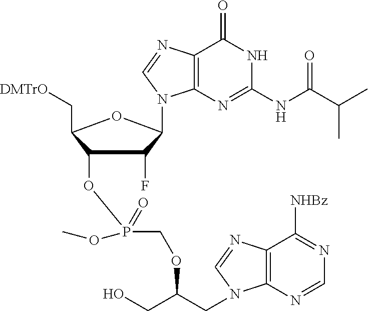

A15. A nucleic acid molecule, where a nucleotide sequence of the nucleic acid molecule includes one or more of the nucleotide monomer according to any one of the technical solutions A1-A12 and/or the nucleotide dimer according to the technical solution A13 or A14.
A16. The nucleic acid molecule according to the technical solution A15, where the nucleic acid is selected from DNA, RNA, and a DNA/RNA hybrid.
A17. The nucleic acid molecule according to the technical solution A15 or A16, where the nucleic acid molecule is single-stranded or double-stranded.
A18. The nucleic acid molecule according to any one of the technical solutions A15-A17, where the nucleic acid molecule is selected from a small interfering RNA (siRNA) and a short hairpin RNA (shRNA).
A19. A double-stranded RNA molecule, including a sense strand and an antisense strand, where each strand has 14 to 30 nucleotides, and the antisense strand includes one or more of the nucleotide monomer according to any one of the technical solutions A1-A12 and/or one or more of the nucleotide dimer according to the technical solution A13 or A14.
A20. The double-stranded RNA molecule according to the technical solution A19, where the sense strand and the antisense strand each has 20 to 25 nucleotides; preferably, a length of the sense strand is 20 nucleotides; preferably, the length of the sense strand is 21 nucleotides; preferably, the length of the sense strand is 22 nucleotides; preferably, the length of the sense strand is 23 nucleotides; preferably, the length of the sense strand is 24 nucleotides; preferably, the length of the sense strand is 25 nucleotides; preferably, a length of the antisense strand is 20 nucleotides; preferably, the length of the antisense strand is 21 nucleotides, preferably, the length of the antisense strand is 22 nucleotides; preferably, the length of the antisense strand is 23 nucleotides, preferably, the length of the antisense strand is 24 nucleotides; and preferably, the length of the antisense strand is 25 nucleotides.
A21. The double-stranded RNA molecule according to the technical solution A19 or A20, where the nucleotide monomer is located at any one or more positions on the sense strand of the antisense strand, including but not limited to any one or more positions of positions 1-30 of the 5′ end of the antisense strand and/or positions 1-30 of the 5′ end of the sense strand, preferably any one or more positions of positions 2-8 of the 5′ end of the antisense strand, preferably position 6 or 7 of the 5′ end of the antisense strand, and more preferably position 7 of the 5′ end of the antisense strand.
A22. The double-stranded RNA molecule according to any one of the technical solutions A19-A21, where the nucleotide dimer is located at any one or more positions on the sense strand or the antisense strand, including but not limited to any one or more positions of positions 1-30 of the 5′ end of the antisense strand and/or positions 1-30 of the 5′ end of the sense strand, preferably positions 2-9 of the 5′ end of the antisense strand, preferably positions 6 and 7 of the 5′ end of the antisense strand, and preferably positions 7 and 8 of the 5′ end of the antisense strand.
A23. The double-stranded RNA molecule according to any one of the technical solutions A19-A22, where the double-stranded RNA exhibits enhanced stability compared with double-stranded RNA having a same sequence but not including the nucleotide monomer according to any one of the technical solutions A1-A12 or the nucleotide dimer according to the technical solution A13 or A14.
A24. The double-stranded RNA molecule according to the technical solution A23, where the double-stranded RNA has a denaturation temperature ranging from about 40° C. to about 80° C., preferably about 55° C. to 67° C.
A25. The double-stranded RNA molecule according to any one of the technical solutions A19-A24, where the double-stranded RNA exhibits reduced off-target toxicity compared with double-stranded RNA having a same sequence but not including the nucleotide monomer according to any one of the technical solutions A1. A12 or the nucleotide dimer according to the technical solution A13 or A14.
A26. The double-stranded RNA molecule according to any one of the technical solutions A19-A25, where the double-stranded RNA exhibits enhanced effectiveness compared with double-stranded RNA having a same sequence but not including the nucleotide monomer according to any one of the technical solutions A1-A12 or the nucleotide dimer according to the technical solution A13 or A14.
A27. The double-stranded RNA molecule according to any one of the technical solutions A19-A26, where the antisense strand has a sequence that is fully complementary to the sense strand and a target mRNA, and has an ability to induce degradation of the target mRNA

A28. The double-stranded RNA molecule according to the technical solution A27, where the target mRNA is encoded by an endogenous gene or encoded by a pathogen gene.
A29. The double-stranded RNA molecule according to any one of the technical solutions A19-A28, where the sense strand and/or the antisense strand includes 3′ and/or 5′ over-hang ends.
A30. The double-stranded RNA molecule according to any one of the technical solutions A19-A29, where the double-stranded RNA is further coupled to a ligand, and preferably, the ligand includes one or more GalNAc(s).
A31. A pharmaceutical composition, including the double-stranded RNA molecule according to any one of the technical solutions A19-A30 and a pharmaceutically acceptable carrier or excipient.
A32. A kit, including the double-stranded RNA molecule according to any one of the technical solutions A19-A30.
A33. A method for inhibiting expression of a target gene in a cell, including a step of introducing the double-stranded RNA molecule according to any one of the technical solutions A19-A30 into the cell.
A34. A method for inhibiting expression of a target gene in a cell, including expressing the double-stranded RNA molecule according to any one of the technical solutions A19-A30 in the cell.

In another aspect, the present invention relates to the following technical solutions:
B1. A nucleotide dimer as shown in Formula III:

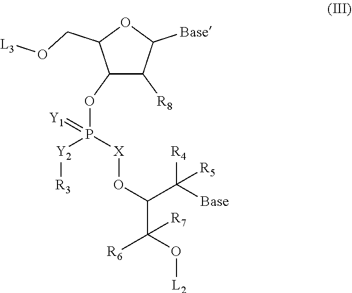

where,
L₂ is H, P₂, or a chemical bond of a phosphate P atom at the 5′ end of ribose that is connected to another nucleotide or oligonucleotide,
L₃ is H, P₃, or a chemical bond of a phosphate P atom at the 2′ or 3′ end of ribose that is connected to another nucleotide or oligonucleotide;
X is a chemical bond or CR₁R₂;
Y₁ is O, S, or NR;
Y₂ is O, S, or a chemical bond;
R₁, R₂, R₄, R₅, R₆ and R₇ are independently selected from H, D, halogen, CN, C₁₋₆ alkyl, C₁₋₆ haloalkyl, C₂₋₆ alkenyl, C₂₋₆ alkynyl, C₃₋₁₀ cycloalkyl, 3- to 10-membered heterocyclyl, C₆₋₁₀ aryl, or 5- to 14-membered heteroaryl, and are optionally substituted with 1, 2, 3, 4, 5, 6, 7, 8 or more R′(s);
R₃ is selected from H, C₁₋₆ alkyl, C₁₋₆ haloalkyl, C₂₋₆ alkenyl, C₂₋₆ alkynyl, C₃₋₁₀ cycloalkyl, 3- to 10-membered heterocyclyl, C₆₋₁₀ aryl, or 5- to 14-membered heteroaryl, and is optionally substituted with 1, 2, 3, 4, 5, 6, 7, 8 or more R′(s);
R₈ is selected from H, OH, halogen, C₁₋₆ alkyl, C₁₋₆ haloalkyl, or C₁₋₆ alkoxyl, preferably fluorine or methoxyl, and preferably H;
Base is H, a modified or unmodified base, or a leaving group,
Base′ is H, a modified or unmodified base, or a leaving group, preferably modified or unmodified A, U, T, O, and C;
P₂ and P₃ are independently selected from a hydroxyl protective group, preferably a silane protective group, an acyl protective group, or an ether protective group, more preferably trimethylsilyl (TMS), triethylsilyl (TES), dimethylisopropylsilyl (DMIPS), diethylisopropylsilyl (DEIPS), tert-butyldimethylsilyl (TBDMS), tert-butyldiphenylsilyl (TBDPS), triisopropylsilyl (TIPS),
acetyl (Ac), chloroacetyl, dichloroacetyl, trichloroacetyl, trifluoroacetyl (TFA), benzoyl, p-methoxybenzoyl, 9-fluorenylmethoxycarbonyl (Fmoc), allyloxycarbonyl (Alloc), 2,2,2-trichloroethoxycarbonyl (Troc), carbobenzoxy (Cbz), tert-butoxycarbonyl (Boc), phenylmethyl (Bn), p-methoxybenzyl (PMB), allyl, triphenylmethyl (Tr), di-p-methoxytriphenylmethyl (DMTr), methoxymethyl (MOM), phenoxymethyl (BOM), 2,2,2-trichloroethoxymethyl, 2-methoxyethoxymethyl (MEM), methylthiomethyl (MTM), or p-methoxybenzyloxymethyl (PMBM);
R is selected from H, C₁₋₆ alkyl, or C₁₋₆ haloalkyl;
R′ is selected from D, halogen, CN, C₁₋₆ alkyl, C₁₋₆ haloalkyl, C₂₋₆ alkenyl, C₂₋₆ alkynyl, C₃₋₁₀ cycloalkyl, 3- to 10-membered heterocyclyl, C₆₋₁₀ aryl, 5- to 14-membered heteroaryl, —OR^{a}, —OC(O)R^{a}, —C(O)R^{a}, —C(O)OR^{a}, —C(O)NR^{a}R^{b}, —S(O)ₙR^{a}, —S(O)ₙOR^{a}, —S(O)ₙNR^{a}R^{b}, —NR^{a}R^{b}, —NR^{a}C(O)R^{b}, NR^{a}—C(O)OR^{b}, —NR^{a}—S(O)ₙR^{b}, or —NR^{a}C(O)NR^{a}R^{b};
n is independently 1 or 2; and
R^{a} and R^{b} are independently selected from H, C₁₋₆ alkyl, C₂₋₆ alkenyl, C₂₋₆ alkynyl, C₃₋₁₀ cycloalkyl, 3- to 10-membered heterocyclyl, C₆₋₁₀ aryl, or 5- to 14-membered heteroaryl; or the R^{a} and the R^{b} as well as nitrogen atoms connected thereto form 3- to 10-membered heterocyclyl.
B2. The nucleotide dimer according to the technical solution B1, where the L₃ is a chemical bond of hydroxyl at the 2′ or 3′ end of ribose that is connected to another nucleotide or oligonucleotide, and the L₃ is preferably a chemical bond of hydroxyl at the 3′ end of ribose that is connected to another nucleotide or oligonucleotide.
B3. The nucleotide diomer according to the technical solution B1 or B2, where the L₂ is H, or a chemical bond of a phosphate P atom at the 5′ end of ribose that is connected to another nucleotide or oligonucleotide; the L₂ is preferably H; and the L₂ is preferably a chemical bond of a phosphate P atom at the 5′ end of ribose that is connected to another nucleotide or oligonucleotide.
B4. The nucleotide dimer according to any one of the technical solutions B1-B3, where the X is a chemical bond.
B5. The nucleotide dimer according to any one of the technical solutions B1-B4, where the X is CR₁R₂, preferably CH₂.
B6. The nucleotide dimer according to any one of the technical solutions B1-B5, where the Y, is O.
B7. The nucleotide dimer according to any one of the technical solutions B1-B6, where the Y is O
B8. The nucleotide dimer according to any one of the technical solutions B1-B7, where the R₁, the R₂ the R₄, the R₅, the R₆ and the R₇ are independently selected from H, D, halogen, CN, C₁₋₆ alkyl, C₁₋₆ haloalkyl, C₂₋₆ alkenyl, C₂₋₆ alkynyl, C₂₋₆ cycloalkyl, 4- to 7-membered heterocyclyl, C₆₋₁₀ aryl, and 5- to 6-membered heteroaryl, preferably H. D, halogen, CN, C₁₋₆ alkyl, C₁₋₆ haloalkyl, C₂₋₆ alkenyl, or C₂₋₆ alkynyl, preferably H. D, halogen, or C₁₋₆ alkyl, and more preferably H or D.
B9. The nucleotide dimer according to any one of the technical solutions B1-B8, where the R₃ is selected from H, C₁₋₆ alkyl, C₁₋₆ haloalkyl, C₂₋₆ alkenyl, C₂₋₆ alkynyl, C₃₋₁₀ cycloalkyl, 3- to 10-membered heterocyclyl, C₆₋₁₀ aryl, or 5- to 14-membered heteroaryl, preferably H, C₁₋₆ alkyl, or C₁₋₆ haloalkyl.
B10. The nucleotide dimer according to any one of the technical solutions B1-B9, where the Base is a modified or unmodified base, preferably modified or unmodified A, U, T, G, and C, and more preferably modified or unmodified G or C.
B11. The nucleotide dimer according to any one of the technical solutions B1-B9, where the Base is a leaving group that is preferably selected from halogen, hydroxyl, —OCOR″, —OTs, or —ONO₂, where R″ is C₁₋₆ alkyl, C₂₋₆ alkenyl, C₂₋₆ alkynyl, C₃₋₁₀ cycloalkyl, 3- to 10-membered heterocyclyl, C₁₋₆ aryl, or 5- to 14-membered heteroaryl; preferably, the Base is halogen or —OTs; and more preferably, the Base is —OTs.
B12. The nucleotide dimer according to any one of the technical solutions B1-B11, having one of the following structures:

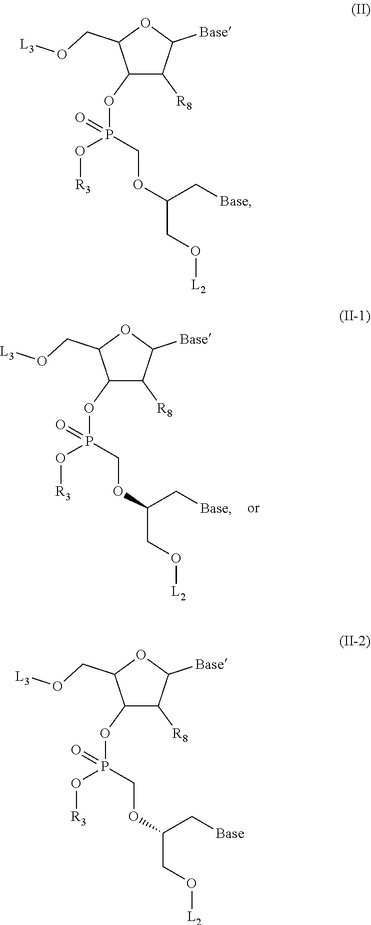

where,
L₂ is H, P₂, or a chemical bond of a phosphate P atom at the 5′ end of ribose that is connected to another nucleotide or oligonucleotide;
L₃ is H, P₃, or a chemical bond of a phosphate P atom at the 2′ or 3′ end of ribose that is connected to another nucleotide or oligonucleotide;
R₃ is selected from H, C₁₋₆ alkyl, or C₁₋₆ haloalkyl;
R₈ is selected from H, OH, halogen, C₁₋₆ alkyl, C₁₋₆ haloalkyl, or C₁₋₆ alkoxyl, preferably fluorine or methoxyl, and preferably H,
Base is a modified or unmodified base, preferably modified or unmodified A, U, T, O, and C, and more preferably modified or unmodified G or C; and
Base′ is H, a modified or unmodified base, or a leaving group, preferably modified or unmodified A, U, T, G, and C.

In another aspect, the present invention relates to the following technical solutions:
C1. A nucleotide dimer as shown in Formula (A),

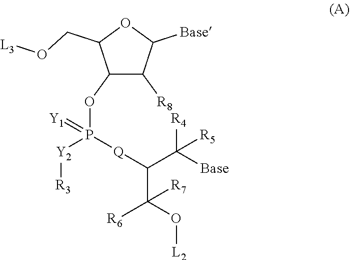

where,
L₂ is H or P₂;
L₃ is H or P₃.
Q is —X— or —X—O—;
X is a chemical bond, —(CR₁R₂)ₘ—, or —CR₁═CR₂—;
Y₁ is O, S, or NR;
Y₂ is O, S, or a chemical bond;
R₁, R₂, R₄, R₅, R₆ and R₇ are independently selected from H, D, halogen, CN, C₁₋₆ alkyl, C₁₋₆ haloalkyl, C₂₋₆ alkenyl, or C₂₋₆ alkynyl, and are optionally substituted with 1, 2, 3, 4, or 5 of R′(s);
R₃ is selected from H, C₁₋₆ cyanoalkyl, C₁₋₆ alkyl, C₁₋₆ haloalkyl, C₂₋₆ alkenyl, C₂₋₆ alkynyl, C₃₋₁₀ cycloalkyl, 3- to 7-membered heterocyclyl, C₆₋₁₀ aryl, or 5- to 10-membered heteroaryl, and is optionally substituted with 1, 2, 3, 4, or 5 R′(s);
R₈ is selected from H, D, OH, halogen, C₁₋₆ alkyl, C₁₋₆ haloalkyl, or C₁₋₆ alkoxyl;
Base and Base′ are independently selected from H, a modified or unmodified base, or a leaving group;
P₂ is selected from a reactive phosphorus group, preferably —P(OCH₂CH₂CN)(N(iPr)₂);
P₃ is selected from a hydroxyl protective group, preferably DMTr;
R is selected from H, C₁₋₆ alkyl, or C₁₋₆ haloalkyl;
R′ is selected from D, halogen, OH, CN, NH₂, C₁₋₆ alkyl, C₁₋₆ haloalkyl, C₂₋₄ alkenyl, C₂₋₆ alkynyl, C₃₋₁₀ cycloalkyl, 3- to 10-membered heterocyclyl, C₆₋₁₀ aryl, or 5- to 14-membered heteroaryl; and m is selected from 1, 2, 3, 4, or 5.
C2. The nucleotide dimer according to the technical solution C1, where,
the L₂ is P₂;
the L₃ is P₃;
the Q is —X— or —X—O—;
the X is a chemical bond, —(CR₁R₂)ₘ—, or —CR₁═CR₂—;
the Y is O or S;
the Y₂ is O or a chemical bond,
the R₁, the R₂, the R₄, the R₅, the R₆ and the R₇ are independently selected from H, D, halogen, CN, C₁₋₆ alkyl, or C₁₋₆ haloalkyl, preferably H, and are optionally substituted with 1, 2, or 3 R′(s);
the R₃ is selected from H, C₁₋₆ alkyl, C₁₋₆ cyanoalkyl, C₁₋₆ haloalkyl, C₂₋₆ alkenyl, or C₂₋₆ alkynyl, and is optionally substituted with 1, 2, or 3 R′(s);
the R₈ is selected from H, OH, halogen, or C₁₋₆ alkoxyl, preferably fluorine or methoxyl, and preferably fluorine;
the Base and the Base′ are independently selected from H, a modified or unmodified base, or a leaving group, and preferably, the Base and the Base′ are independently selected from

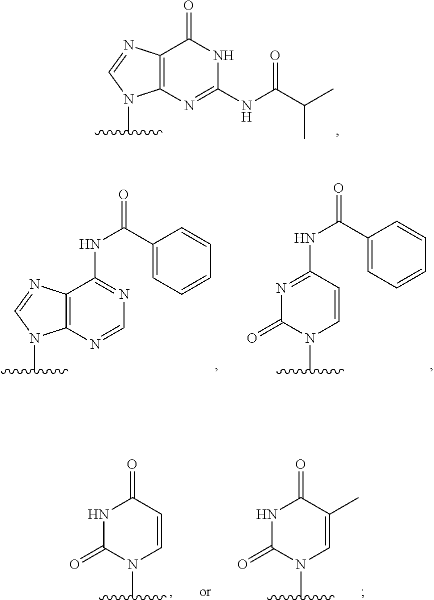

the P₂ is selected from a reactive phosphorus group, preferably —P(OCH₂CH₂CN)(N(iPr)₂);
the P₃ is selected from a hydroxyl protective group, preferably DMTr;
the R′ is selected from D, halogen, OH, CN, NH₂, C₁₋₆ alkyl, or C₁₋₆ haloalkyl; and
the m is selected from 1, 2, or 3.
C3. The nucleotide dimer according to the technical solution C1 or C2, where,
the L₂ is P₂;
the L₃ is P₃;
the Q is —X— or —X—O—;
the X is —CH₂, —CH₂CH₂—, or —CH═CH—;
the Y₁ is O;
the Y₂ is O;
the R₁, the R₂, the R₄, the R₅, the R₆ and the R₇ are independently selected from H, halogen, or C₁₋₄ alkyl, preferably H;
the R₃ is selected from H, C₁₋₄ alkyl, or C₁₋₄ cyanoalkyl, preferably methyl or cyanoethyl;
the R₈ is selected from halogen or C₁₋₄ alkoxyl, preferably fluorine or methoxyl, and preferably fluorine,
the Base and the Base′ are independently selected from

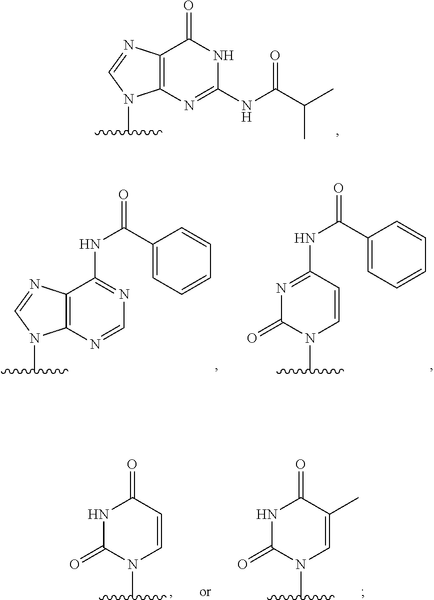

the P₂ is selected from a reactive phosphorus group, preferably —P(OCH₂CH₂CN)(N(iPr)₂); and
the P₃ is selected from a hydroxyl protective group, preferably DMTr.
C4. The nucleotide dimer according to any one of the technical solutions C1-C3, where the Q is —CH₂—O—.
C5. The nucleotide dimer according to any one of the technical solutions C1-C3, having the following structures:

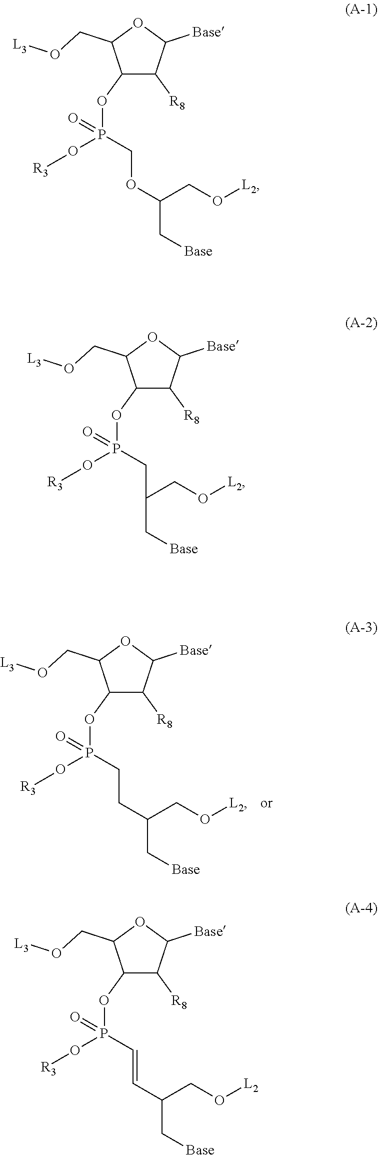

where,
R₃ is methyl or cyanoethyl; and
various other groups are as defined according to any one of the technical solutions C1-C₃.
C6. The nucleotide dimer according to any one of the technical solutions C1-C5, having the following structures:

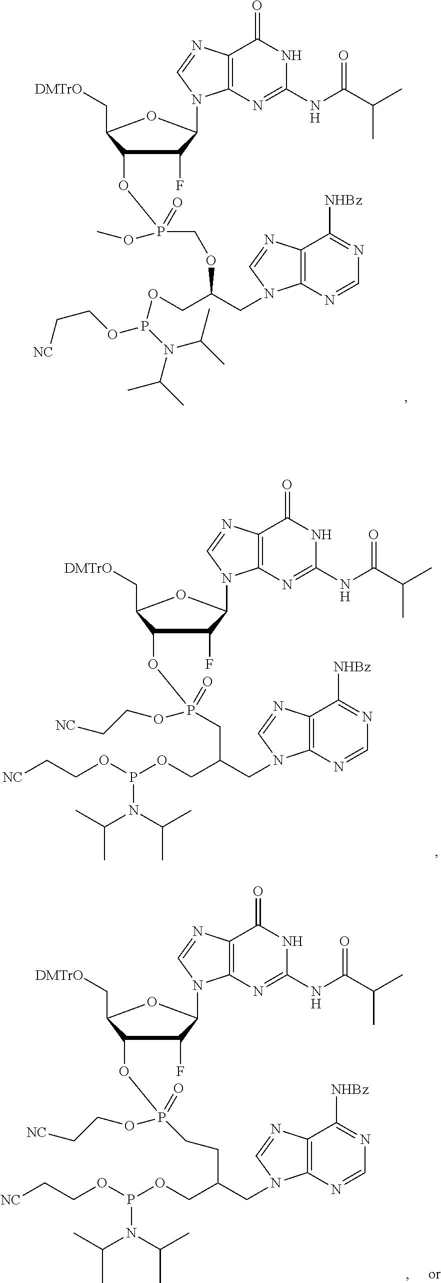

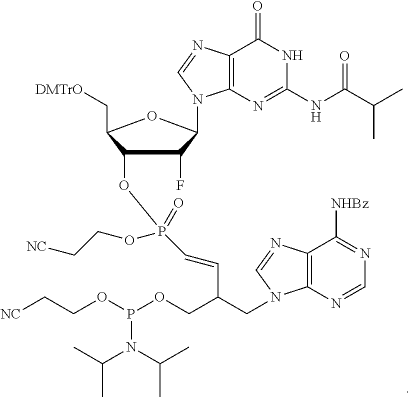

C7. A double-stranded RNA molecule or a pharmaceutically acceptable salt, tautomer or stereoisomer thereof, including a sense strand and an antisense strand, where each strand has 14 to 30 nucleotides, the antisense strand has a sequence that is fully complementary to the sense strand and a target mRNA, and has an ability to induce degradation of the target mRNA, and the antisense strand includes one or more of a nucleotide monomer as shown in Formula (IV):

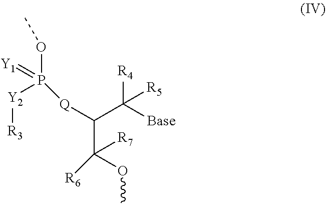

where,
the nucleotide monomer is connected from [symbol] to [symbol] the order of 5′=>3′;
various groups are as defined according to any one of the technical solutions C1-C6;
preferably, the nucleotide monomer is selected from:

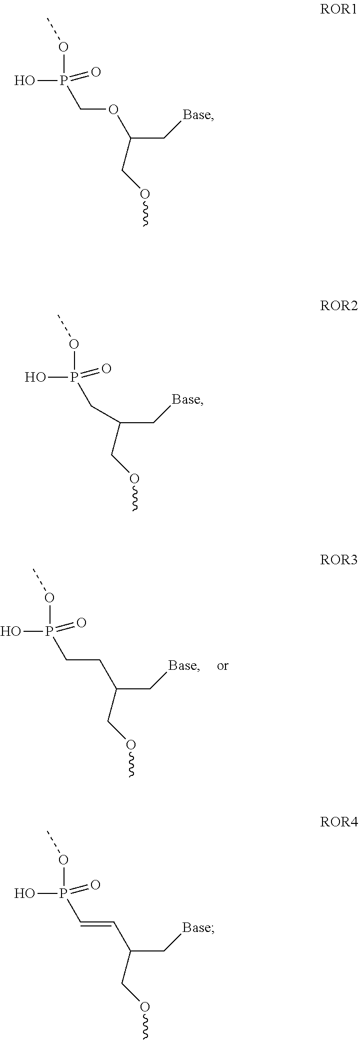

and
Base is selected from

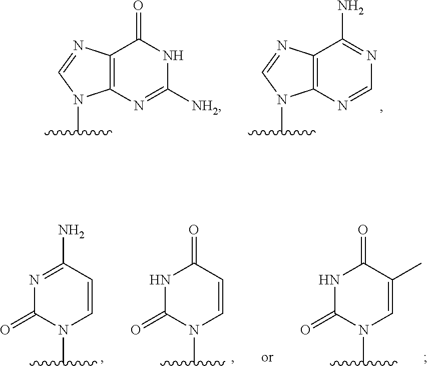

C8. The double-stranded RNA molecule according to the technical solution C7, where the double-stranded RNA molecule is prepared from the nucleotide dimer according to any one of the technical solutions C1-C6.
C9. The double-stranded RNA molecule according to the technical solution C7 or C8, where the sense strand and the antisense strand each has 20 to 30 nucleotides.
C10. The double-stranded RNA molecule according to any one of the technical solutions C7-C9, where the nucleotide monomer is located at positions 2-8 of the 5′ end of the antisense strand, preferably position 6 or 7, and more preferably position 7.
C11. The double-stranded RNA molecule according to any one of the technical solutions C7-C10, where the double stranded RNA exhibits enhanced stability compared with double-stranded RNA having a same sequence but not including the nucleotide monomer according to the technical solution C7.
C12. The double-stranded RNA molecule according to the technical solution C11, where the double-stranded RNA has a denaturation temperature ranging from about 40° C. to about 80° C., preferably about 55° C. to 67° C.
C13. The double-stranded RNA molecule according to any one of the technical solutions C7-C12, where the double-stranded RNA exhibits reduced off-target toxicity compared with double-stranded RNA having a same sequence but not including the nucleotide monomer according to the technical solution C7.
C14. The double-stranded RNA molecule according to any one of the technical solutions C7-C13, where the double-stranded RNA exhibits enhanced effectiveness compared with double-stranded RNA having a same sequence but not including the nucleotide monomer according to the technical solution C7
C15. The double stranded RNA molecule according to any one of the technical solutions C7-C14, where the sense strand and/or the antisense strand includes a 3′ and/or 5′ over-hang end
C16. The double-stranded RNA molecule according to any one of the technical solutions C7-C15, where the double stranded RNA is further coupled to a ligand, and preferably, the ligand includes one or more GalNAc(s).
C17 A preparation method for the double stranded RNA molecule according to any one of the technical solutions C7-C16, where the double-stranded RNA molecule is prepared from the nucleotide dimer according to any one of the technical solutions C1-C6.
C18 A nucleic acid molecule, where a nucleotide sequence of the nucleic acid molecule includes one or more of the nucleotide monomer according to the technical solution C7.
C19. The nucleic acid molecule according to the technical solution C18, where the nucleic acid is selected from DNA, RNA, and a DNA/RNA hybrid.
C20. The nucleic acid molecule according to the technical solution C19, where the nucleic acid molecule is single-stranded or double-stranded.
C21. The nucleic acid molecule according to any one of the technical solutions C18-C20, where the nucleic acid molecule is selected from a small interfering RNA (siRNA) and a short hairpin RNA (shRNA).
C21. A pharmaceutical composition, including the double-stranded RNA molecule according to any one of the technical solutions C7-C16 and a pharmaceutically acceptable carrier or excipient.
C22. A kit, including the double-stranded RNA molecule according to any one of the technical solutions C7-C16.
C23. A method for inhibiting expression of a target gene in a cell, including a step of introducing the double-stranded RNA molecule according to any one of the technical solutions C7-C16 into the cell.
C24. A method for inhibiting expression of a target gene in a cell, including expressing the double-stranded RNA molecule according to any one of the technical solutions C7-C16 in the cell.

### DESCRIPTION OF THE EMBODIMENTS

The following embodiments are merely used for illustrating the present invention and are not intended to limit the scope of the present invention.

### Example 1: Synthesis of a Nucleotide Dimer of a Compound 14

### Synthesis Route:

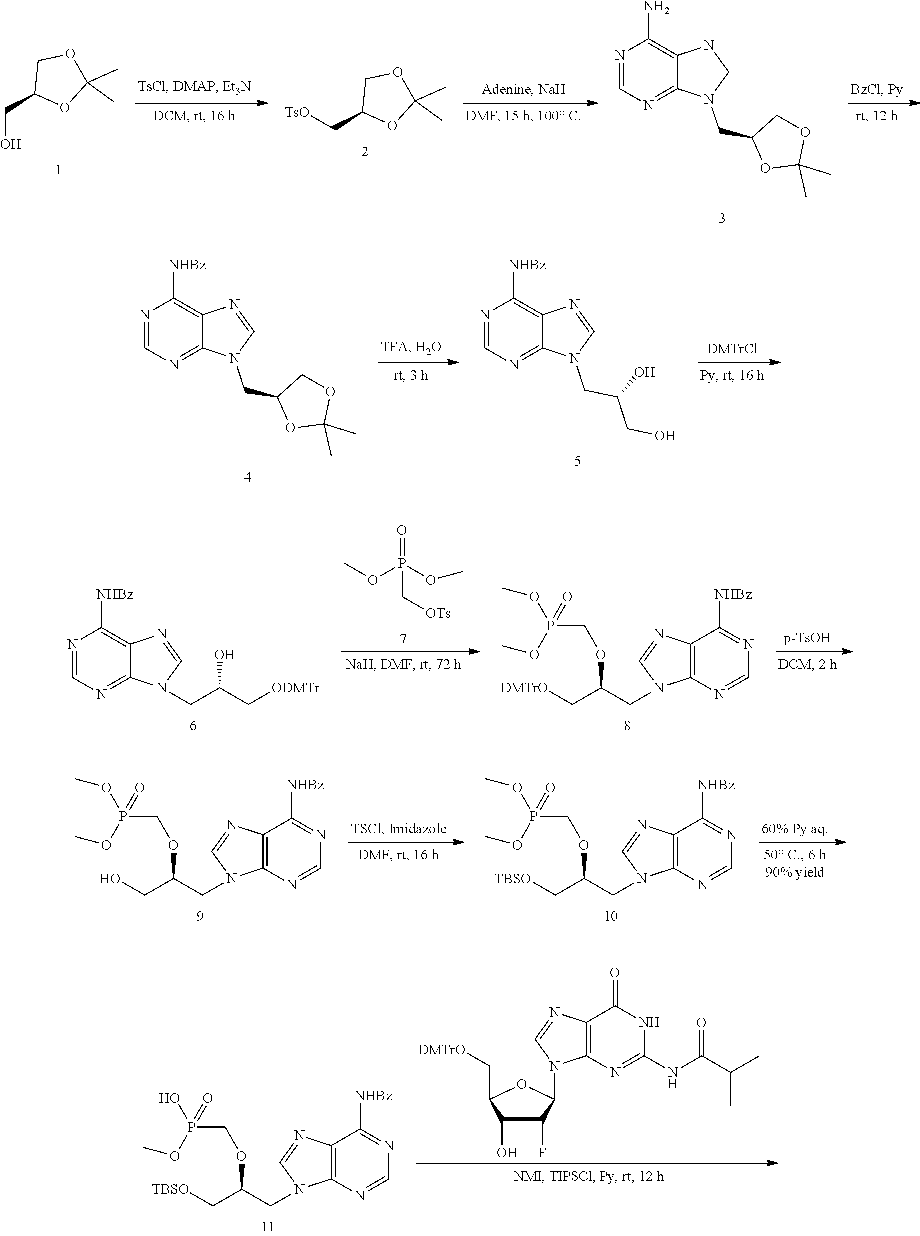

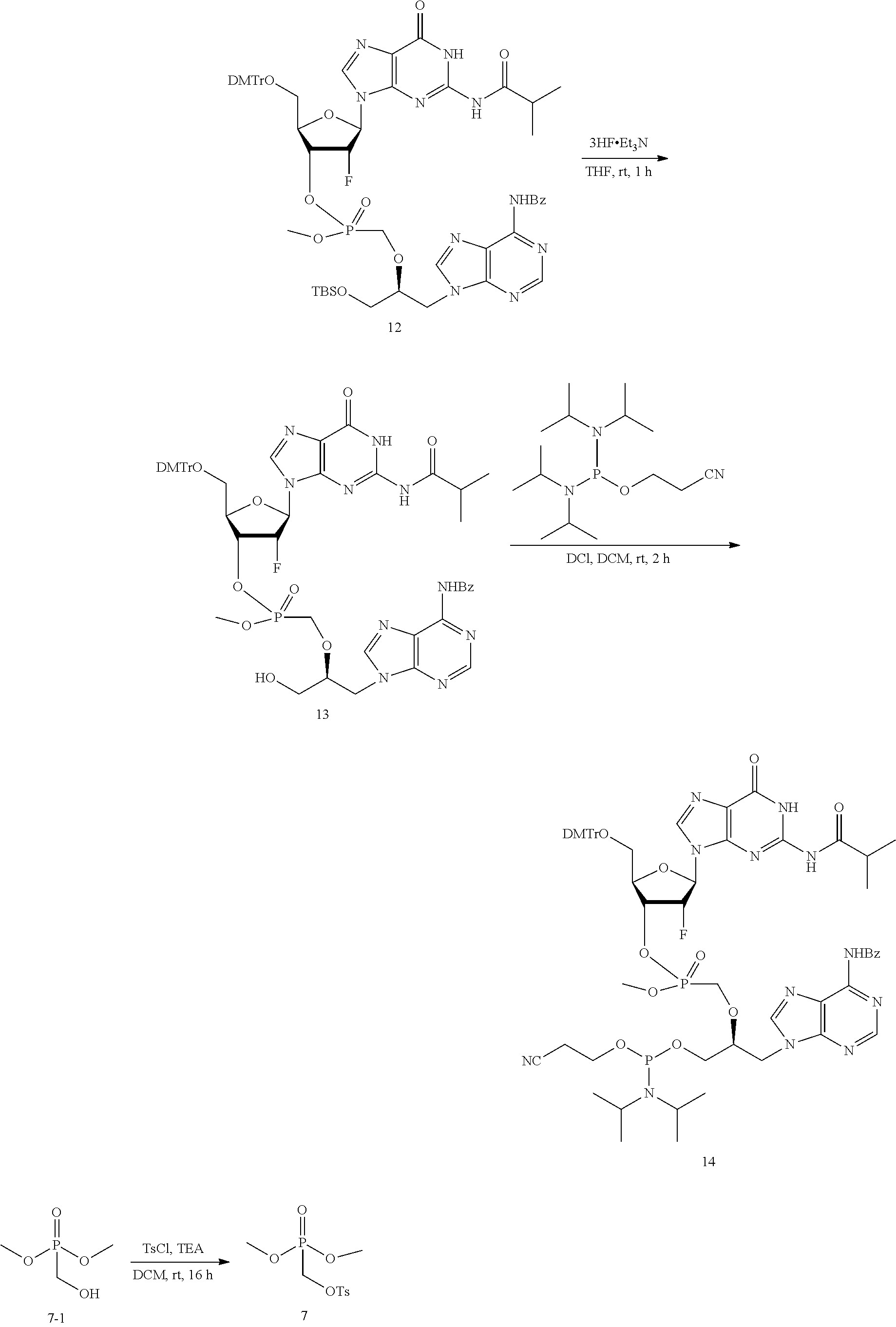

### Experimental Steps

### 1. Synthesis of a Compound 2

A compound 1 (25 g, 189 165 mmol) was dissolved in 250 mL of dichloromethane, then p-toluenesulfonyl chloride (54.09 g, 283.747 mmol), triethylamine (47.85 g, 472.912 mmol) and 4-dimethylaminopyridine (2.31 g, 18.916 mmol) were sequentially added, and a reaction mixture was stirred overnight at room temperature. According to monitoring by TLC, reaction raw materials were completely reacted. Then, the reaction system was diluted with dichloromethane (300 ml), and sequentially washed with a saturated NaHCO₃ aqueous solution (3×300 mL), citric acid (1×200 mL) and saturated salt water (300 mL). An organic phase was dried with anhydrous sodium sulfate, filtration was carried out, and a filtrate was concentrated under reduced pressure to obtain a yellow oily compound 2 (42.0 g, 286.34 mmol, 77.54%).

m/z=286.2 [M+H]⁺

### 2. Synthesis of a Compound 3

Adenine (23.78 g, 176.015 mmol) was dissolved in 600 mL of N,N-dimethylformamide under the protection of argon, sodium hydride (5.28 g, 220 018 mmol) was added in batches under an ice water bath, and a reaction mixture was heated to 100° C. and stirred for 2 hours. Then, the compound 2 (42 g, 146.679 mmol) was dissolved in 200 mL of N,N-dimethylformamide, added dropwise to the above reaction system, and continuously stirred overnight at 100° C. According to monitoring by TLC, reaction raw materials were completely reacted. Then, a reaction was quench with methanol (20 mL), a reaction solvent was spin-dried under reduced pressure, and a crude product was purified by chromatography with a 100- to 200-mesh silica gel column (methanol:dichloromethane=0-10%, 30 min) to obtain a white solid compound 3 (1 g, 249.27 mmol, 49.23%).

m/z=250.2 [M+H]⁺.

### 3. Synthesis of a Compound 4

The compound 3 (18 g, 72.211 mmol) was dissolved in 200 mL of anhydrous pyridine, and benzoyl chloride (20.30 g, 144.422 mmol) was added under the condition of room temperature and stirred overnight. According to monitoring by TLC, reaction raw materials were completely reacted. Then, an aminomethanol solution (7 M, 200 mL) was added and stirred for 10 minutes, the reaction system was spin-dried under reduced pressure, and a crude product was purified by chromatography with a 100- to 200-mesh silica gel column (methanol:dichloromethane=0-10%, 30 min) to obtain a yellow solid compound 4 (16 g, 31.513 mmol, 43.6494).

m/z=354.2 [M+H]⁺.

### 4. Synthesis of a Compound 5

The compound 4 (25 g, 70.740 mmol) was dissolved in a mixed solution of trifluoroacetic acid (180 mL) and water (60 mL), and the reaction system was stirred at room temperature for 3 hours. According to monitoring by TLC, reaction raw materials were completely reacted. Then, the reaction system was spin-dried under reduced pressure, and a crude product was purified by chromatography with a 100- to 200-mesh silica gel column (methanol:dichloromethane: 0-15%, 30 min) to obtain a white solid compound 5 (16 g, 51.066 mmol, 72.19%).

m/z=314.0 [M+H]⁺

### 5. Synthesis of a Compound 6

The compound 5 (3.9 g, 12.447 mmol) was dehydrated with anhydrous pyridine for three times and then dissolved in anhydrous pyridine (40 mL), 4,4-dimethoxytriphenylchloromethane (5.06 g, 14.937 mmol) was added at room temperature under the protection of argon, and the reaction system was stirred overnight at room temperature. According to detection by LC-MS, raw materials were completely converted. The reaction system was diluted with ethyl acetate (200 mL) and washed with water for 2 times with 50 mL each time, an organic phase was dried with anhydrous sodium sulfate, filtration was carried out, a filtrate was concentrated and dried under reduced pressure, and a crude product was purified by chromatography with a 100- to 200-mesh silica gel column (ethyl acetate:petroleum ether: 0-50%, 40 min) to obtain a yellow solid compound 6 (6.2 g, 10.077 mmol, 80.96%).

m/z=616.2 [M++H]⁺.

### 6. Synthesis of a Compound 7

Dimethyl hydroxymethylphosphonate (9.3 g, 66.395 mmol) was dissolved in dichloromethane (100 mL), a reaction solution was cooled to 0° C., triethylamine (11.997 mL, 86.314 mmol) and p-toluenesulfonyl chloride (13.92 g, 73.035 mmol) were slowly added under the protection of argon, and a reaction mixture was stirred overnight at room temperature. According to monitoring by LC-MS, reaction raw materials were completely reacted. A reaction solution was diluted with dichloromethane, washed with water and dried with anhydrous sodium sulfate, filtration was carried out, a filtrate was concentrated under reduced pressure, and a crude product was purified by chromatography with a 100- to 200-mesh silica gel column (methanol:dichloromethane=0-20%, 40 min) to obtain a light yellow solid compound 7 (11 g, 37.382 mmol, 56.30%).

m/z: 294.8 [M+H]⁺

### 7. Synthesis of a Compound 8

The compound 6 (400 mg, 0.650 mmol) was dissolved in anhydrous N,N-dimethylformamide (20 mL) under the protection of nitrogen, sodium hydride (46.77 mg, 1.950 mmol) was added under an ice water bath, and a reaction mixture was stirred for 30 minutes under the ice water bath. The compound 7 (382.35 mg, 1.300 mmol) was dissolved in anhydrous N,N-dimethylformamide (2 mL) and then slowly added dropwise to the above reaction system. After a reaction mixture was stirred at room temperature for 72 hours, raw materials were completely converted according to monitoring by LC-MS. A reaction was quenched with ice water, dilation was carried out with dichloromethane (100 mL), an organic phase was washed with ice water and dried with anhydrous sodium sulfate, filtration was carried out, and a filtrate was concentrated under reduced pressure to obtain a yellow solid crude compound 8 (510 mg).

m/z=294.8 [M+H]⁺.

### 8. Synthesis of a Compound 9

The compound 8:500 mg, 0.678 mmol) was dissolved in methanol/dichloromethane (1:3, 8 mL), and p-methylbenzene sulfonic acid (64.46 mg, 0.339 mmol) was added at room temperature. A reaction mixture was stirred at room temperature for 2 hours and then concentrated under reduced pressure, and a crude product was purified by chromatography with a 100- to 200-mesh silica gel column (methanol:dichloromethane=0-10%, 40 min) to obtain a white solid compound 9 (145 mg, 49.14%).

¹H NMR (400 MHZ, DMSO-d₆): δ 11.14 (s, 1H), 8.74 (s, 1H), 8.39 (3, 1H), 8.03-8.06 (m, 2H), 7.52-7.68 (m, 3H), 4.99-5.03 (m, 1H), 4.31-4.53 (m, 2H), 3.83-4.12 (m, 3H), 3.57 (s, 3H), 3.53 (s, 3H), 3.46 (s, 2H).

### 9. Synthesis of a Compound 10

The compound 9 (650 mg, 1.49 mmol) was dissolved in anhydrous N,N-dimethylformamide (5 mL) under the protection of nitrogen, and imidazole (304.8 mg, 4.48 mmol) and tert-butyldimethylchlorosilane (336.2 mg, 2.24 mmol) were added at room temperature, respectively. Reaction compounds were stirred overnight at room temperature. According to monitoring by LC-MS, raw materials were completely converted. A reaction mixture was quenched with ice water and extracted with ethyl acetate (3×50 mL), an organic phase was washed with salt water and dried with anhydrous sodium sulfate, filtration was carried out, a filtrate was concentrated under reduced pressure, and a crude product was purified by chromatography with a 100- to 200-mesh silica gel column (methanol dichloromethane=0-3%, 40 min) to obtain a white-like solid compound 10 (710 mg, 1.29 mmol, 86%).

m/z=550.4 [M+H]⁺.

### 10. Synthesis of a Compound 11

The compound 10 (710 mg, 1.29 mmol) was dissolved in a pyridine aqueous solution (3:2, 20 mL), and a reaction mixture was stirred at 50° C. for 6 hours. According to monitoring by LC-MS, raw materials were completely converted. A reaction solation was concentrated under reduced pressure, and a crude product was directly used in a next reaction without purification.

m/z=536.2 [M+H]⁺.

### 11. Synthesis of a Compound 12

The crude compound 11 (450 mg) was dissolved in anhydrous pyridine (S mL) under the protection of argon, triisopropylchlorosilane (763.94 rog, 2.522 mmol) was added under an ice bath, and the reaction system was stirred under the condition of room temperature for IS minutes. Then, 5′-O-(4,4′-dimethoxytriphenylmethyl)-N2-isobutyryl-2 fluorodeoxyguanosine (580.26 mg, 0.883 mmol) and N-methylimidazole (345.15 mg, 4.204 mmol) were dissolved in anhydrous pyridine (5 mL) and slowly added to the above reaction system. A reaction mixture was stirred overnight at room temperature. According to monitoring by LC-MS, raw materials were completely converted. A reaction was quenched with a sodium bicarbonate aqueous solution (1.0 M), the reaction mixture was concentrated under reduced pressure, and a crude product was purified by a reverse-phase chromatographic column (5 M ammonium bicarbonate aqueous solution/acetonitrile) to obtain a yellow solid compound 12 (480 mg, 63%).

m/z=1175.6 [M+H]⁺.

¹H NMR (400 MHZ, DMSO-d₆): δ 8.65 (3, 1H), 8.26 (s, 1H), 8.08-8.20 (m, 1H), 7.82-7.96 (m, 2H), 7.55-7.62 (m, 1H), 7.45-7.50 (m, 2H), 7.06-7.28 (m, 9H), 7.70-7.82 (m, 4H), 6.16-6.22 (m, 1H), 5.68-5.85 (m, 1H), 5.30-5.45 (m, 1H), 3.86-4.42 (m, 5H), 3.52-3.76 (m, 7H), 3.22-3.46 (m, 9H), 2.03 (8, 2H), 1.05-1.11 (m, 6H), 0.81-0.83 (m, 9H), 0.00-0.04 (m, 6H).

¹⁹F NMR (DMSO-d₆): δ −204.82, −204.53.

³¹P NMR (DMSO-d₆): δ 22.94, 22.72.

### 12. Synthesis of a Compound 13

The compound 12 (200 mg, 0.170 mmol) was dissolved in anhydrous tetrahydrofuran (10 mL) under the protection of nitrogen, a tetrahydrofuran (2 mL) solution of triethylamine hydrofluorate (137.26 mg, 0.851 mmol) was slowly added under an ice water bath, and the reaction system was heated to room temperature to carry out a reaction for 1 hour. According to monitoring by LC-MS, reaction raw materials were completely converted. The reaction system was diluted with ethyl acetate and quenched with water, an organic phase was dried with anhydrous sodium sulfate after being separated, filtration was carried out, a filtrate was concentrated under reduced pressure, and a crude product was purified by reversed-phase preparative chromatography (preparation conditions: chromatographic column: XBridge Shield RP18 OBD column, 19*150 mm, 5 μm; mobile phase A: water (10 mmol/L NH₄HCO₃), and mobile phase B: ACN; flow rate: 20 ml/min; gradient: 50% B to 60% B within 5.5 min, and 60% B; wavelength: 254/210 nm; and RT1 (min): 4.5, 5.1) to obtain a white solid compound 13 (69 mg, 0.065 mmol, 38.21%).

m/z=1062.3 [M+H]⁺.

¹H NMR (400 MHZ, DMSO-de): δ 8.70 (s, 1H), 8.31 (s, 1H) 8.15 (s, 1H), 7.98-8.00 (m, 2H), 7.46-7.62 (m, 3H), 7.16-7.40 (m, 9H), 6.70-6.65 (m, 4H), 6.25-6.20 (m, 1H), 5.68-5.90 (m, 1H), 5.38-5.46 (m, 1H), 4.96-5.02 (m, 1H), 3.82-4.48 (m, 12H), 3.70 (s, 2H), 3.25-3.46 (m, 9H), 1.11-1.13 (m, 6H).

¹⁹F NMR (400 MHZ, DMSO-d₆) δ −204.54.

³¹P NMR (400 MHZ, DMSO-d₆): δ 23.17.

### 13. Synthesis of a Compound 14

Bis(diisopropylamino)(2-cyanoethoxy)phosphine (23.45 mg, 0.078 mmol) was dehydrated with ultra-dry acetonitrile for three times first, and then the compound 13 (55 mg, 0.052 mmol) was dehydrated with ultra-dry acetonitrile for three times. The bis(diisopropylamino)(2-cyanoethoxy phosphine (23.45 mg, 0.078 mmol) was dissolved in anhydrous dichloromethane (1.5 mL) under the protection of argon, and 4,5-dicyanoimidazole (4.90 mg, 0.041 mmol) was added at room temperature. Then, an anhydrous dichloromethane (1 mL) solution of the compound 13 (55 mg, 0.052 mmol) was added to the above reaction system. After a reaction mixture was stirred at room temperature for 2 hours, raw materials were completely converted according to monitoring by LC-MS. The reaction system was added dropwise to an icy sodium bicarbonate aqueous solution and extracted with dichloromethane, an organic phase was dried with anhydrous sodium sulfate, filtration was carried out, a filtrate was concentrated under reduced pressure, and a crude product was purified by reversed-phase chromatography (with pure water and acetonitrile, and about 70% of a product was produced). A collection solution was extracted with dichloromethane and dried with anhydrous sodium sulfate, filtration was carried out, and a filtrate was concentrated under reduced pressure to obtain a white solid compound 14 (30 mg, 0.024 mmol, 45.89%).

m/z=1261.7 [M+H]⁺.

¹H NMR (400 MHZ, CD₃CN): δ 8.55 (s, 1H), 8.09 (3, 1H), 7.72-7.89 (m, 3H), 7.41-7.53 (m, 3H), 7.03-7.08 (m, 5H), 6.91-7.01 (m, 4H), 6.50-6.73 (m, 4H), 6.16-6.22 (m, 1H), 5.95-6.00 (m, 1H), 5.62-5.72 (m, 1H), 4.44-4.48 (m, 1H), 4.22-4.26 (m, 1H), 4.12-4.18 (m, 1H), 3.91-4.08 (m, 2H), 3.59-3.88 (m, 10H), 3.50-3.54 (m, 2H), 3.27-3.35 (m, 4H), 2.98-3.02 (m, 1H), 2.53-2.64 (m, 3H), 2.00-2.10 (m, 1H), 1.05-1.10 (m, 18H).

³¹P NMR (400 MHZ, CD₃CN) δ 148.69, 148.57, 23.54, 23.50.

¹⁹F NMR (400 MHZ, CD₃CN) δ −203.63, −203.69.

### Example 2 Preparation of siRNA

The siRNA of the present invention is prepared by a solid-phase phosphoramidite method known in the field. The method can specifically refer to, for example, PCT publication No. WO2016081444 and WO2019105419, and is briefly described below.

### 1. Synthesis of a Sense Strand (SS Strand)

Through the solid-phase phosphoramidite synthesis method, with a blank CPO solid phase carrier or a solid phase carrier connected with L96 as an initial cycle, nucleoside monomers were connected one by one in a direction from 3′ to 5′ according to a nucleotide arrangement sequence of a sense strand. Connection of each of the nucleoside monomers includes reactions in four steps including deprotection, coupling, capping, and oxidation or thiolation Synthesis conditions of oligonucleotide with a synthesis scale of 5 umol are as follows:
the nucleoside monomers were provided by a 0.05 mol/L acetonitrile solution, and the reaction in each step was carried out under a same condition, namely a temperature of 25° C.; the deprotection was carried out with a 3% trichloroacetic acid-dichloromethane solution, and the deprotection was carried out for 3 times; the coupling reaction was carried out with a 0.25 mol/L ETT-acetonitrile solation as an activator, and the coupling reaction was carried out for 2 times; the capping was carried out with 10% acetic anhydride-acetonitrile and pyridine/N-methylimidazole/acetonitrile (10:14:76, v/v/v), and the capping was carried out for 2 times; the oxidation was carried out with 0.05 mol/L iodine/tetrahydrofuran/pyridine/water (70/20/10, v/v/v), and the oxidation was carried out for 2 times; and the thiolation was carried out with 0.2 mol/L acetonitrile/3-methylpyridine (1/1, v/v) of PADS, the thiolation was carried out for 2 times.

### 2. Synthesis of an Antisense Strand (AS Strand)

Through the solid-phase phosphoramidite synthesis method, with a blank CPG solid phase carrier as an initial cycle, nucleoside monomers or nucleotide dimers of the present invention were connected one by one in a direction from 3′ to 5′ according to a nucleotide arrangement sequence of an antisense strand. Connection of each of the nucleoside monomers or the nucleotide dimers of the present invention includes reactions in four steps including deprotection, coupling, capping, and oxidation or thiolation. Synthesis conditions of 5 umol oligonucleotide of the antisense strand are the same as those of the sense strand.

### 3. Purification and Annealing of Oligonucleotide

### 3.1. Ammonolysis

A synthesized solid phase carrier (sense strand or antisense strand) was added to a 5 mL centrifuge tube, 3% diethylamine/ammonia water (v/v) was added, and a reaction was carried out under a constant-temperature water bath at 35° C. for 55° C.) for 16 hours for 8 hours). Filtration was carried out, the solid phase carrier was washed with ethanol/water for three times with 1 mL each time, a filtrate was centrifuged and concentrated, and a crude product was purified.

### 3.2. Purification

Methods for purification and desalination are well known to persons in the field. For example, a strong anion filler column can be used, a sodium chloride-sodium hydroxide system was used for elution and purification, a product was collected in a tube, a gel filler purification column can be used for desalination, and an elution system was pure water.

### 3.3. Annealing

According to the following table, the sense strand (SS strand) and the antisense strand (AS strand) were mixed at a molar ratio (SS strand/AS strand=1/1.05), heated to 70-95° C. in a water bath pot, maintained for 3-5 min, and then naturally cooled to room temperature, and the system was freeze-dried to obtain a product.

An siRNA sequence used in the present invention is as follows:

| Double | Sequence (5′→3′) |
|---|---|
| strand | A first one is an SS strand sequence, |
| number | and a second one is an AS strand sequence. |
| DR005709 | UmsGmsAmCmAmAmGfAfAfUmCmCmUmCmAmCmAmAmUm |
| | AmsUfsUmGfUmGfAmGfGmAfUmUfCmUfUmGfUmCfAmsAfsCm |
| DR005737 | UmsGmsAmCmAmAmGfAfAfUmCmCmUmCmAmCmAmAmUm |
| | AmsUfsUmGfUmGf(ROR1-A)GfGmAfUmUfCmUfUmGfUmCfAmsAfsCm |
| DR000359 | UmsUmsCmUmCmAmGfUfGfCmUmCmUmCmCmUmAmCmUm |
| | AmsGfsUmAfGmGfAmGfAmGfCmAfCmUfGmAfGmAfAmsUfsUm |
| DR005751 | UmsUmsCmUmCmAmGfUfGfCmUmCmUmCmCmUmAmCmUm |
| | AmsGfsUmAfGmGf(ROR1-A)GfAmGfCmAfCmUfGmAfGmAfAmsUfsUm |
| DR002220 | CmsAmsGmUmGfUmUfCfUfUmGmCmUmCmUmAmUmAmAm-L96 |
| | UmsUfsAmUmAmGfAmGmCmAmAmGmAmAfCmAfCmUmGmsUmsUm |
| DR005760 | CmsAmsGmUmGfUmUfCfUfUmGmCmUmCmUmAmUmAmAm-L96 |
| | UmsUfsAmUmAmGf(ROR1-A)GmCmAmAmGmAmAfCmAfCmUmGmsUmsUm |

Herein, meanings of various abbreviation are as follows:

Distribution of A, U, G and C represents natural adenine ribonucleotide, uracil ribonucleotide, guanine ribonucleotide, and cytosine ribonucleotide.
m represents that an adjacent nucleotide on the left side is 2′-OCH₃ modified nucleotide. For example, Am, Um, Gm and Cm represent 2′-OCH₃ modified A, U, G, and C.
f represents that an adjacent nucleotide on the left side is 2′-F modified nucleotide. For example, Af, Uf, Gf and Cf represent 2′-F modified A, U, G, and C, respectively.
“s” or “s-” represents that two nucleotides and/or delivery vectors that are adjacent to each other are connected by thiophosphate.

L96 represents a GalNAc delivery vector having the following structure well known in the field, where [symbol] represents a position connected with siRNA by a phosphate group or a thiophosphate group, with reference to, for example, PCT publication No. WO2009073809 and WO2009082607.

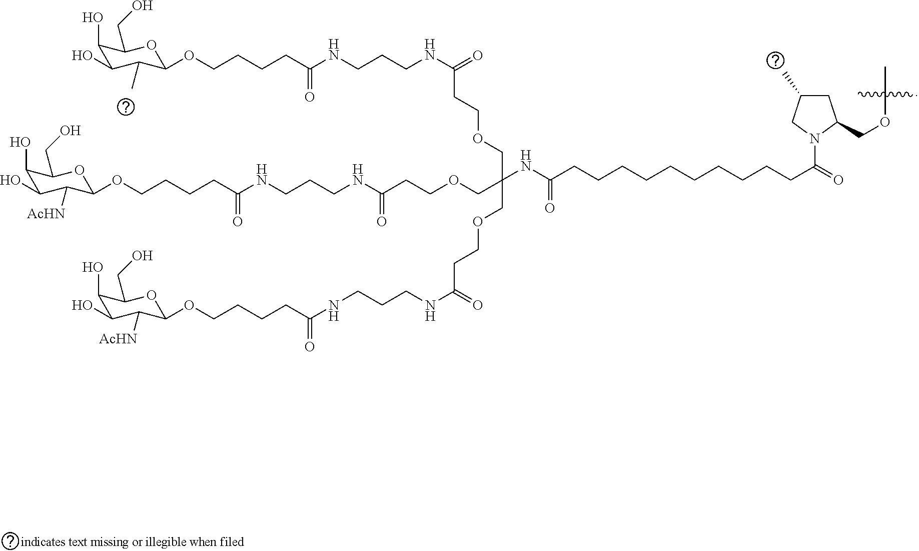 [SMILES: *OC[C@@H]1C[C@@H](C)CN1C(=O)CCCCCCCCCCC(=O)NC(COCCC(=O)NCCCNC(=O)CCCCOC1OC(CO)C(O)C(O)C1C)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

ROR1 represents a nucleotide substitute of the above structure, where Base can be any base, for example, ROR1-A represents that the Base is adenine.

### Example 3 Detection of On-Target and Off-Target Activity of an siRNA Compound psi-CHECK2

### 1. Preparation of Plasmids

On-target plasmid: A corresponding on-target plasmid of an antisense strand was designed according to a sequence of a compound, a psiCHECK2 GSCM recombinant plasmid was prepared by Sangon Biotech (Shanghai) Co., Ltd., and the recombinant plasmid was diluted to 1,000 ng/μL for later use.

Off-target plasmid: A corresponding off-target plasmid of the antisense strand was designed according to the sequence of the compound, a psiCHECK2 OSSM-5Hits recombinant plasmid was prepared by Sangon Biotech (Shanghai) Co., Ltd., and the recombinant plasmid was dilated to 1,000 ng/μL for later use.

### 2. Cell Transfection

HEK293A cells (Nanjing cobioer, article No. CBP60436) were used, and 100 μL of a cell resuspension was spread on a 96-well plate at a cell volume of 8×10³ cells/well.

On the next day, a complete culture medium in wells was first sucked away and then changed into an Opti-MEM culture medium at 80 μL/well, and starvation treatment was carried out for about 1.5 h. Plasmid mixture: A preparation volume in each well was as follows: plasmid at 0.01 μl/well and Opti-MEM at 8.99 (L/well.

Lipo mixture: Lipo 2000 (Lipofectamine™ 2000 transfection reagent, Thermo, 11668019) was dilated with Opti-MEM and subjected to standing at room temperature for 5 min. A preparation volume of the Lipo mixture was as follows: Lipo at 0.2 μL/well and Opti-MEM at 9.8 μL/well.

22 μL of the prepared Lipo mixture, 22 μL of the compound and 19.8 μL of the plasmid mixture were separately loaded to a same corresponding well, named Well A, evenly mixed by blowing and beating, incubated at room temperature for 20 min and then subjected to co-transfection. Finally, a well A mixture was added to the cells in each well at 20 μl/well, and 80 μL of original Opti-MEM was added to make a final volume of each well reach 100 μL. After culture was carried out in an incubator containing 5% of CO₂ at 37° C. for 4 h, each well was supplemented with 100 μL of a DMEM culture medium containing 20% of fetal bovine serum. After culture was carried out in the incubator containing 5% of CO₂ at 37° C. for 24 h, detection was carried out.

### 3. Detection of Results

Before an experiment, well mixed Dual-Glo® Luciferase (Dual-Glo® Luciferase Assay System, Promega, E2940) was remelted, equalized to room temperature and then added with DMEM at a ratio of 1:1 m each tube to prepare a substrate I, which was prepared for immediate use. Dual-Glo® Stop & Glo® Buffer was remelted, equalized to room temperature and then mixed with Dual-Glow Stop & Glow Substrate at a ratio of 100:1 to prepare a substrate II, which was prepared for immediate use.

An original culture medium in a 96-well culture plate was sucked away by a vacuum pump.

150 μL of the substrate I was added to each well and incubated on a shaker at room temperature for 10 min.

120 μL of the substrate I was transferred to a 96-well ELISA plate, and a Firefly chemiluminescence value on a microplate reader (Tecan, Infinite 200) was read.

Then, 60 μL of the substrate II was added to each well and incubated on a shaker at room temperature for 10 min A Renilla chemilumenescence value on the microplate reader was read.

### 4. Analysis and Processing of Data

The fluorescence activity is measured by the microplate reader, collected Renilla signals are normalized in accordance with a Firefly signal standard, an inhibitory effect of siRNA is obtained by comparing unprocessed results (residual inhibitory activity), and a calculation process is shown as follows:
normalized Ren/Fir ratio: Ratio=Renilla (renilla luciferase)/Firefly (firefly luciferase).

Residual inhibitory rate: An average value of (Ratio_{siRNA}/Ratio_{control})*100% of 2 duplicated wells is calculated, where Ratio_{control} is an average Ratio value of 2 duplicated wells of control wells (without siRNA), a Ratio_{siRNA}/Ratio_{control} value of the 2 duplicated wells is calculated, respectively, and then an average value is calculated as a residual inhibitory rate.

Mapping: Mapping is carried out by using Graphpad Prism.

Half maximal inhibitory concentration (IC50): In this experiment, mapping is carried out based on Top and Bottom, and an IC50 value is obtained according to a formula Y=Bottom+(Top−Bottom)/(1+10\hat{ }((LogIC50−X)*HillSlope)), where Y=50, and X=log (concentration).

In an on-target activity detection experiment, an HEK293A (Nanjing cobioer, article No. CBP60436) cell line was selected and transfected with a psiCHECK2-GSCM recombinant plasmid, the compound with an initial concentration of 10 nM was selected and then diluted by 3 times to obtain 11 concentration points (10 nM, 3.33 nM, 1.11 nM, 0.37 nM, 0.123 nM, 0.041 nM, 0.0136 nM, 0.0045 nM, 0.00152 nM, 0.000508 nM, 0.000169 nM), and the activity of the siRNA compound was screened. Experimental results are shown in Table 1.

TABLE 1
Experimental results of on-target activity detection of psi-CHECK2

| | | | | | | | | | | | | | IC50 |
|---|---|---|---|---|---|---|---|---|---|---|---|---|---|
| | Double | 10 | 3.3333 | 1.1111 | 0.37037 | 0.12346 | 0.04115 | 0.01372 | 0.00457 | 0.00152 | 0.00051 | 0.00017 | (nM) |
| Gene | strand code | nM | nM | nM | nM | nM | nM | nM | nM | nM | nM | nM | value |
| HBV-S | DR005709 | 1.5% | 1.8% | 1.8% | 2.3% | 6.8% | 20.3% | 53.4% | 82.4% | 96.6% | 105.2% | 97.0% | 0.0142 |
| | DR005737 | 3.5% | 4.1% | 3.0% | 2.0% | 2.6% | 6.5% | 20.9% | 65.9% | 93.3% | 111.3% | 109.6% | 0.0053 |
| APOC3 | DR000359 | 0.8% | 0.7% | 0.8% | 1.1% | 3.5% | 12.7% | 37.0% | 72.7% | 94.4% | 99.4% | 99.5% | 0.0092 |
| | DR005751 | 0.6% | 0.6% | 0.6% | 1.2% | 3.6% | 12.3% | 36.8% | 76.8% | 95.7% | 106.6% | 102.7% | 0.0089 |

In an off-target activity detection experiment, an HEK293A (Nanjing cobioer, article No. CBP60436) cell line was selected and transfected with a psiCHECK2-GSCM-5Hits recombinant plasmid, the compound with an initial concentration of 10 nM was selected and then diluted by 3 times to obtain 11 concentration points (10 nM, 3.33 nM, 1.11 nM, 0.37 nM, 0.123 nM, 0.041 nM, 0.0136 nM, 0.0045 nM, 0.00152 nM, 0.000508 nM, 0.000169 nM), and the activity of the siRNA compound was screened. Experimental results are shown in Table 2.

TABLE 2
Experimental results of off-target activity detection of psi-CHECK2

| | | | | | | | | | | | | | Off- |
|---|---|---|---|---|---|---|---|---|---|---|---|---|---|
| | | | | | | | | | | | | | target |
| | | | | | | | | | | | | | IC50 |
| | Double | 10 | 3.3333 | 1.1111 | 0.37037 | 0.12346 | 0.04115 | 0.01372 | 0.00457 | 0.00152 | 0.00051 | 0.00017 | value |
| Gene | strand code | nM | nM | nM | nM | nM | nM | nM | nM | nM | nM | nM | (nM) |
| HBV-S | DR005709 | 3.1% | 4.0% | 4.9% | 15.6% | 60.8% | 100.2% | 115.4% | 110.2% | 110.9% | 113.5% | 108.7% | 0.013 |
| | DR005737 | 43.6% | 55.6% | 89.7% | 113.8% | 125.9% | 114.3% | 120.0% | 114.8% | 117.0% | 118.3% | 112.3% | 3.970 |
| APOC3 | DR000359 | 9.5% | 8.8% | 10.4% | 20.4% | 53.8% | 91.7% | 98.6% | 105.3% | 104.1% | 106.8% | 101.6% | 0.117 |
| | DR005751 | 48.1% | 50.8% | 68.9% | 92.4% | 96.9% | 99.6% | 102.1% | 102.9% | 101.7% | 105.4% | 95.0% | 4.546 |

The results show that siRNA carrying a nucleotide analog monomer of the present invention effectively reduces the off-target activity under the premise of maintaining the target activity.

### Example 4 Verification of Long-Term Efficacy of a Compound in a C57BL/6 Mouse Model

C57BL/6 mice (male, 18-21 g, 6- to 8-week-ok) were randomly grouped with 6 animals in each group according to Table 3. An administration dosage of each animal was calculated according to a body weight, and single administration was carried out by a subcutaneous injection method. An siRNA compound was first prepared into a 1 mg/ml solution (with a 0.9% sodium chloride aqueous solution as a solvent). Before an experiment, the siRNA compound was dissolved in the 0.9% sodium chloride solution and metered to a required solution concentration and a volume. A dosage volume of normal saline and the siRNA compound was 5 mL/kg.

TABLE 3
Grouping of an animal experiment

| Serial | |
|---|---|
| number | Compound-group |
| 1 | Normal saline |
| 2 | DR002220 |
| 3 | DR005760 |

Blood was taken from orbital venous plexus of the mice before administration (recorded as day 0) and 14, 28, 42 and 56 days after the administration, respectively, and mTTR protein in the serum was detected by an ELISA kit (Abcam, ab282297) at various time points. At a last experimental time point, 10 mg of liver was taken and placed in an RNAlater solution, and mRNA of mTTR in the liver was detected.

TABLE 4
Verification results of long-term efficacy of a compound in a C57BL/6 mouse model

| | Day 14 | | Day 28 | | Day 42 | | Day 56 | |
|---|---|---|---|---|---|---|---|---|
| Double | Residual | | Residual | | Residual | | Residual | |
| strand | mTTR | | mTTR | | MTTR | | mTTR | |
| number | protein | SD | protein | SD | protein | SD | protein | SD |
| Normal saline | 100.0% | 10.0% | 100.0% | 8.1% | 100.0% | 6.3% | 100.0% | 4.9% |
| DR002220 | 2.3% | 0.8% | 6.8% | 2.4% | 16.3% | 4.6% | 33.4% | 7.6% |
| DR005760 | 3.4% | 0.4% | 9.4% | 1.0% | 18.2% | 2.8% | 43.2% | 4.4% |

The results show that siRNA carrying a nucleotide analog monomer of the present invention can reduce expression of a target gene in vivo for a long time.

1. A nucleotide dimer as shown in Formula (A), 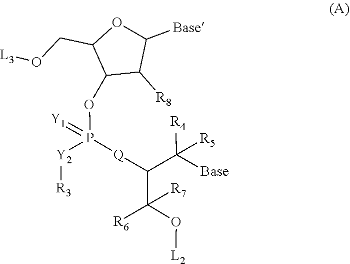
wherein,
L₂ is H or P₂;
L₃ is H or P₃;
Q is —X— or —X—O—;
X is a chemical bond, —(CR₁R₂)ₘ—, or —CR₁═CR₂—;
Y is O, S, or NR;
Y₂ is O, S, or a chemical bond;
R₁, R₂, R₄, R₅, R₆ and R₇ are independently selected from H, D, halogen, CN, C₁₋₆ alkyl, C₁₋₆ haloalkyl, C₂₋₆ alkenyl, or C₂₋₆ alkynyl, and are optionally substituted with 1, 2, 3, 4, or 5 R′(s);
R₃ is selected from H, C₁₋₆ cyanoalkyl, C₁₋₆ alkyl, C₁₋₆ haloalkyl, C₂₋₆ alkenyl, C₂₋₆ alkynyl, C₃₋₇ cycloalkyl, 3- to 7-membered heterocyclyl, C₆₋₁₀ aryl, or 5- to 10-membered heteroaryl, and is optionally substituted with 1, 2, 3, 4, or 5 R′(s);
R₈ is selected from H, D, OH, halogen, C₁₋₆ alkyl, C₁₋₆ haloalkyl, or C₁₋₆ alkoxyl;
Base and Base′ are independently selected from H, a modified or unmodified base, or a leaving group;
P₂ is selected from a reactive phosphorus group, preferably —P(OCH₂CH₂CN)(N(iPr)₂);
P₃ is selected from a hydroxyl protective group, preferably DMTr;
R is selected from H, C₁₋₆ alkyl, or C₁₋₆ haloalkyl;
R′ is selected from D, halogen, OH, CN, NH₂, C₁₋₆ alkyl, C₁₋₆ haloalkyl, C₂₋₆ alkenyl, C₂₋₆ alkynyl, C₃₋₁₀ cycloalkyl, 3- to 10-membered heterocyclyl, C₆₋₁₀ aryl, or 5- to 14-membered heteroaryl; and
m is selected from 1, 2, 3, 4, or 5.

2. The nucleotide dimer according to claim 1, wherein,
the L₂ is P₂;
the L₃ is P₃;
the Q is —X— or —X—O—;
the X is a chemical bond, —(CR₁R₂)ₘ—, or —CR₁═CR₂—;
the Y is O or S;
the Y₂ is O or a chemical bond;
the R₁, the R₂, the R₄, the R₅, the R₆ and the R₇ are independently selected from H, D, halogen, CN, C₁₋₆ alkyl, or C₁₋₆ haloalkyl, preferably H, and are optionally substituted with 1, 2, or 3 R′(s);
the R₃ is selected from H, C₁₋₆ alkyl, C₁₋₆ cyanoalkyl, C₁₋₆ haloalkyl, C₂₋₆ alkenyl, or C₂₋₆ alkynyl, and is optionally substituted with 1, 2, or 3 R′(s);
the R₅ is selected from H, OH, halogen, or C₁₋₆ alkoxyl, preferably fluorine or methoxyl, and preferably fluorine;
the Base and the Base′ are independently selected from H, a modified or unmodified base, or a leaving group, and preferably, the Base and the Base′ are independently selected from 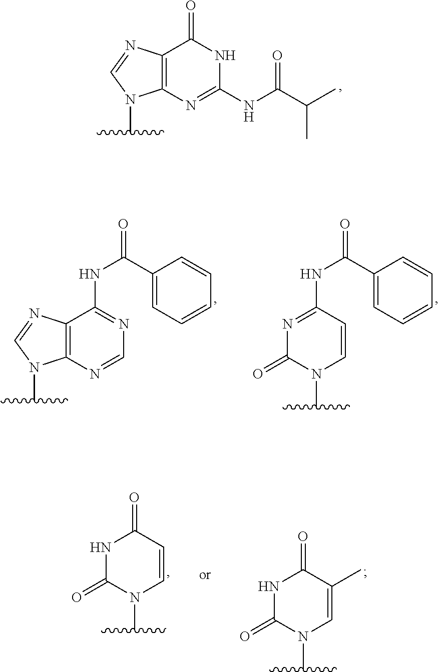
the P₂ is selected from a reactive phosphorus group, preferably —P(OCH₂CH₂CN)(N(iPr)₂);
the P₃ is selected from a hydroxyl protective group, preferably DMTr;
the R′ is selected from D, halogen, OH, CN, NH₂, C₁₋₆ alkyl, or C₁₋₆ haloalkyl; and
the m is selected from 1, 2, or 3.

3. The nucleotide dimer according to claim 1, wherein,
the L₂ is P₂;
the L₃ is P₃;
the Q is —X— or —X—O—;
the X is —CH₂—, —CH₂—CH₂—, or —CH═CH—;
the Y is O;
the Y₂ is O;
the R₁, the R₂, the R₄, the R₅, the R₆ and the R₇ are independently selected from H, halogen, or C₁₋₄ alkyl, preferably H;
the R₃ is selected from H, C₁₋₄ alkyl, or C₁₋₄ cyanoalkyl, preferably methyl or cyanoethyl;
the R₅ is selected from halogen or C₁₋₄ alkoxyl, preferably fluorine or methoxyl, and preferably fluorine;
the Base and the Base′ are independently selected from 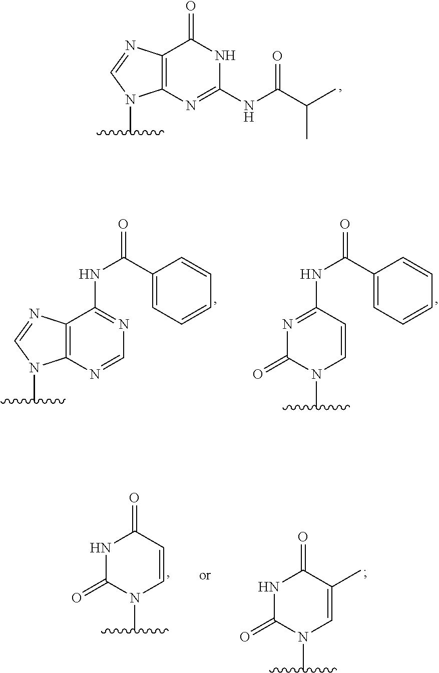
the P₂ is selected from a reactive phosphorus group, preferably —P(OCH₂CH₂CN)(N(iPr)₂); and
the P₃ is selected from a hydroxyl protective group, preferably DMTr.

4. The nucleotide dimer according to claim 1, wherein the Q is —CH₂—O—.

5. The nucleotide dimer according to claim 1, having the following structures: 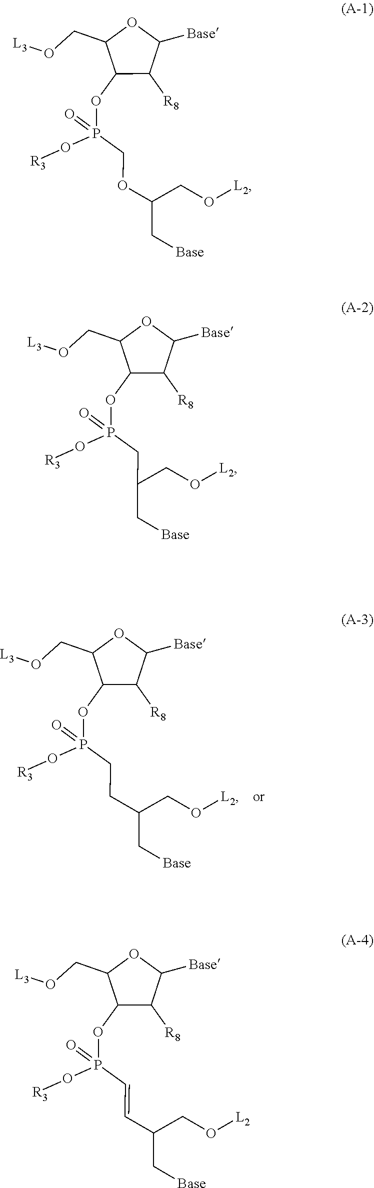
wherein,
R₃ is methyl or cyanoethyl; and
various other groups are as defined according to claim 1.

6. The nucleotide dimer according to claim 5, having the following structures:

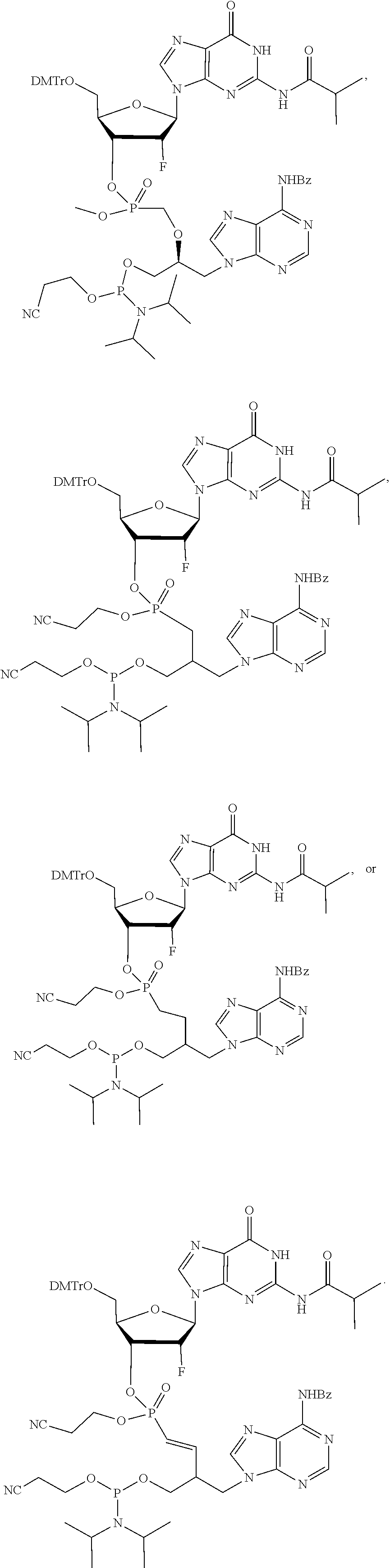

7. A double-stranded RNA molecule or a pharmaceutically acceptable salt, tautomer or stereoisomer thereof, comprising a sense strand and an antisense strand, wherein each strand has 14 to 30 nucleotides, the antisense strand has a sequence that is fully complementary to the sense strand and a target mRNA, and has an ability to induce degradation of the target mRNA, and the antisense strand comprises one or more of a nucleotide monomer as shown in Formula (IV): 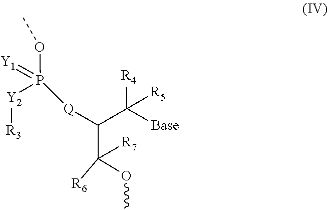
wherein,
the nucleotide monomer is connected from [symbol] to [symbol] in the order of 5′=>3′;
various groups are as defined according to claim 6;
preferably, the nucleotide monomer is selected from: 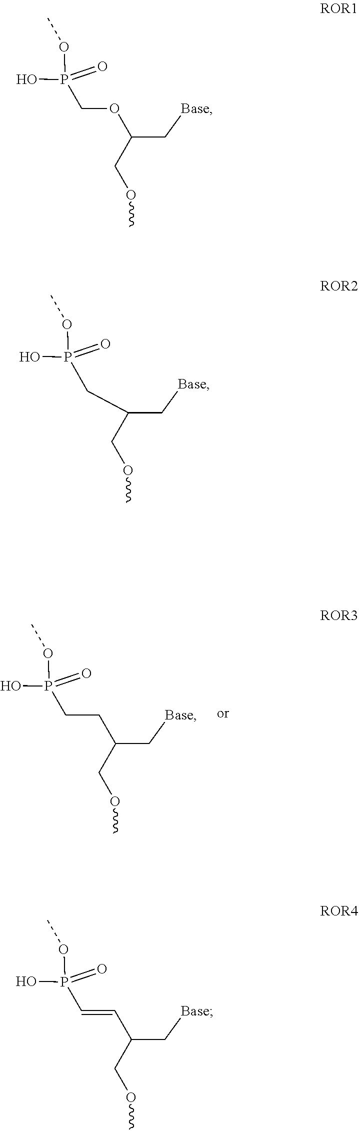
and
Base is selected from

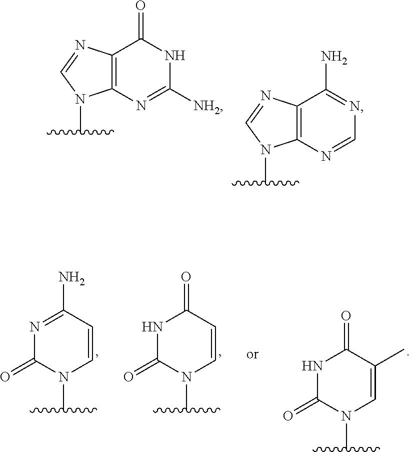

8. A double-stranded RNA molecule or a pharmaceutically acceptable salt, tautomer or stereoisomer thereof, comprising a sense strand and an antisense strand, wherein each strand has 14 to 30 nucleotides, the antisense strand has a sequence that is fully complementary to the sense strand and a target mRNA, and has an ability to induce degradation of the target mRNA, and the antisense strand comprises one or more of a nucleotide monomer as shown in Formula (IV): 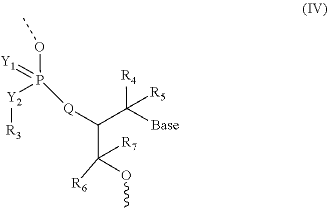
wherein,
the nucleotide monomer is connected from [symbol] to [symbol] in the order of 5′=>3′;
various groups are as defined according to claim 1;
wherein the double-stranded RNA molecule is prepared from the nucleotide dimer according to claim 6.

9. The double-stranded RNA molecule according to claim 7, wherein the nucleotide monomer is located at positions 2-8 of the 5′ end of the antisense strand, preferably position 6 or 7, and more preferably position 7.

10. The double-stranded RNA molecule according to claim 7, wherein the double-stranded RNA is further coupled to a ligand, and preferably, the ligand comprises one or more GalNAc(s).

11. A preparation method for a double-stranded RNA molecule or a pharmaceutically acceptable salt, tautomer or stereoisomer thereof, wherein the double-stranded RNA molecule comprises a sense strand and an antisense strand, each strand has 14 to 30 nucleotides, the antisense strand has a sequence that is fully complementary to the sense strand and a target mRNA, and has an ability to induce degradation of the target mRNA, and the antisense strand comprises one or more of a nucleotide monomer as shown in Formula (IV): 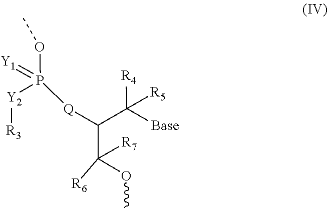
wherein,
the nucleotide monomer is connected from [symbol] to [symbol] in the order of 5′=>3′;
various groups are as defined according to claim 1; wherein the double-stranded RNA molecule is prepared from the nucleotide dimer according to claim 1.

12. A pharmaceutical composition, comprising the double-stranded RNA molecule according to claim 7 and a pharmaceutically acceptable carrier or excipient.

13. A method for inhibiting expression of a target gene in a cell, comprising a step of introducing the double-stranded RNA molecule according to claim 7 into the cell.

14. A double-stranded RNA molecule or a pharmaceutically acceptable salt, tautomer or stereoisomer thereof, comprising a sense strand and an antisense strand, wherein each strand has 14 to 30 nucleotides, the antisense strand has a sequence that is fully complementary to the sense strand and a target mRNA, and has an ability to induce degradation of the target mRNA, and the antisense strand comprises one or more of a nucleotide monomer, wherein the nucleotide monomer is selected from: 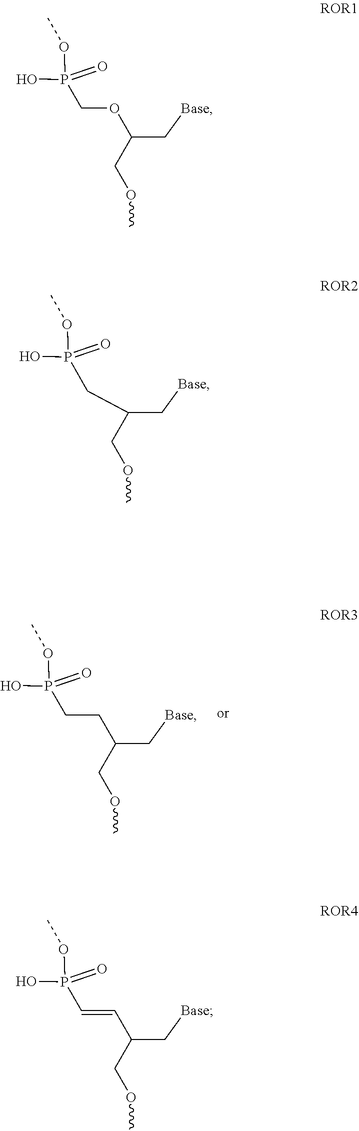
and
Base is selected from 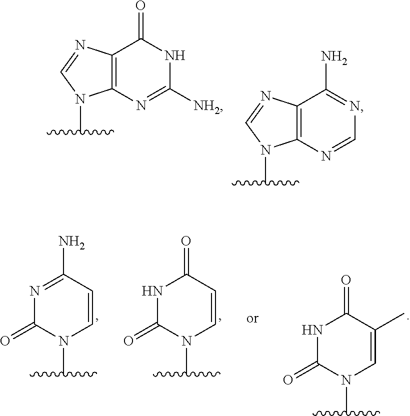
wherein the double-stranded RNA molecule is prepared from the nucleotide dimer according to claim 1.

15. A preparation method for a double-stranded RNA molecule or a pharmaceutically acceptable salt, tautomer or stereoisomer thereof, wherein the double-stranded RNA molecule comprises a sense strand and an antisense strand, each strand has 14 to 30 nucleotides, the antisense strand has a sequence that is fully complementary to the sense strand and a target mRNA, and has an ability to induce degradation of the target mRNA, and the antisense strand comprises one or more of a nucleotide monomer, wherein the nucleotide monomer is selected from: 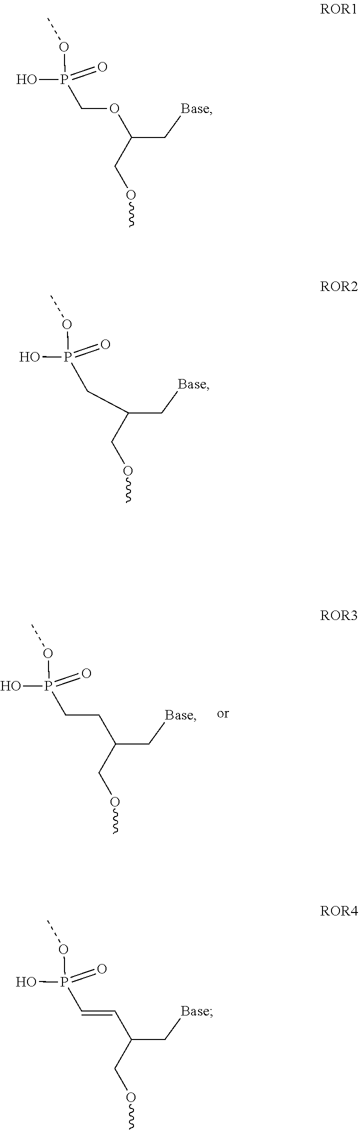
and
Base is selected from 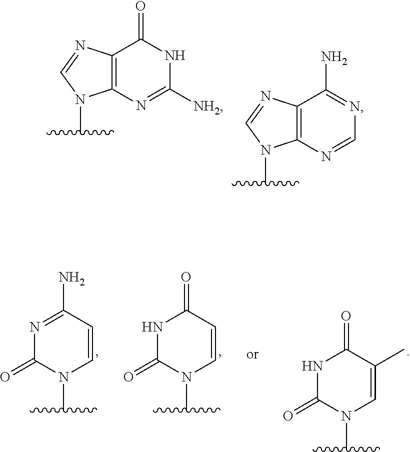
wherein the double-stranded RNA molecule is prepared from the nucleotide dimer according to claim 1.
